(19) United States
(12) Patent Application Publication
(10) Pub. No.: US 2019/0074445 A1
(43) Pub. Date: Mar. 7, 2019

(54) ORGANIC LIGHT-EMITTING DEVICE INCLUDING FLUORESCENT COMPOUND AND FLUORESCENT COMPOUND

(71) Applicants: Samsung Electronics Co., Ltd., Suwon-si (KR); Ewha University - Industry Collaboration Foundation, Seoul (KR)

(72) Inventors: Sooghang IHN, Hwaseong-si (KR); Youngmin YOU, Seoul (KR); Sonam KIM, Seoul (KR); Sinheui KIM, Seoul (KR); Seungyeon YI, Seoul (KR); Sunghan KIM, Seongnam-si (KR); Changho NOH, Suwon-si (KR); Soohwan SUL, Suwon-si (KR); Myungsun SIM, Suwon-si (KR); Hasup LEE, Seoul (KR)

(21) Appl. No.: 16/119,308

(22) Filed: Aug. 31, 2018

(30) Foreign Application Priority Data
Sep. 1, 2017 (KR) 10-2017-0111935
Aug. 28, 2018 (KR) 10-2018-0101449

(51) Int. Cl. H01L 51/00; C07D 311/10; C07C 49/784; C07C 49/786; C07C 49/683

(52) U.S. Cl. CPC H01L 51/0052; C07D 311/10; C07C 49/784; H01L 51/5016; C07C 49/683; H01L 51/0073; C07C 49/786

(57) ABSTRACT

An organic light-emitting device comprising: a first electrode; a second electrode facing the first electrode; and an organic layer disposed between the first electrode and the second electrode, wherein the organic layer comprises an emission layer and a fluorescent compound, wherein the fluorescent compound comprises a ³n-π*-to-¹π-π* energy transition from a ³n-π* excited state to a ¹π-π* excited state, an energy level in a ¹n-π* excited state of the fluorescent compound is greater than an energy level in the ¹π-π* excited state of the fluorescent compound, the fluorescent compound emits a fluorescent light by radiative energy transition of an exciton in the ¹π-π* excited state to a ground state, and the energy level in the ¹n-π* excited state, the energy level in the ¹π-π* excited state, and the energy level in the ³n-π* excited state are each independently calculated by using a time dependent-Density Functional Theory method.

### CROSS-REFERENCE TO RELATED APPLICATION

This application claims priority to and the benefit of Korean Patent Applications Nos. 10-2017-0111935, filed on Sep. 1, 2017 and 10-2018-0101449, filed on Aug. 28, 2018, in the Korean Intellectual Property Office, and all the benefits accruing therefrom under 35 U.S.C. § 119, the disclosures of which are incorporated herein in their entireties by reference.

### BACKGROUND

### 1. Field

The present disclosure relates to an organic light-emitting device including a fluorescent compound satisfying a predetermined condition, and to the fluorescent compound.

### 2. Description of the Related Art

Organic light-emitting devices (OLEDs) are self-emission devices that have wide viewing angles, high contrast ratios, and short response times. In addition, OLEDs have excellent brightness, driving voltage, and response speed characteristics, and produce full-color images.

As an example, an organic light-emitting device includes an anode, a cathode, and an organic layer that is disposed between the anode and the cathode and includes an emission layer. A hole transport region may be disposed between the anode and the emission layer, and an electron transport region may be disposed between the emission layer and the cathode. Holes provided from the anode may move toward the emission layer through the hole transport region, and electrons provided from the cathode may move toward the emission layer through the electron transport region. The holes and electrons are recombined in an emission layer region to produce excitons. These excitons transition from an excited state to a ground state, thereby generating light.

Meanwhile, emission using a triplet exciton may include phosphorescence and thermally activated delayed fluorescence (TADF), but such phosphorescence and TADF have disadvantages in that compound deterioration can be rapid due to a relatively longer exciton lifetime. Furthermore, a phosphorescent compound is often a metal-containing compound using iridium, platinum, or the like, and in this regard, use of such a phosphorescent compound can be expensive.

Thus, there remains a need to develop cost-efficient OLEDs having high luminance and high emission efficiency.

### SUMMARY

Provided are organic light-emitting devices including a fluorescent compound satisfying a predetermined condition, and the fluorescent compound therein.

Additional aspects will be set forth in part in the description which follows and, in part, will be apparent from the description, or may be learned by practice of the presented embodiments.

An aspect provides an organic light-emitting device including:
a first electrode;
a second electrode facing the first electrode; and
an organic layer disposed between the first electrode and the second electrode, wherein the organic layer includes an emission layer and a fluorescent compound,
wherein the fluorescent compound includes a ³n-π*-to-¹π-π* energy transition from a ³n-π* excited state to a ¹π-π* excited state,

an energy level in a ¹n-π* excited state of the fluorescent compound is greater than an energy level in the ¹π-π* excited state of the fluorescent compound,

the fluorescent compound emits a fluorescent light by radiative energy transition of an exciton in the ¹π-π* excited state to a ground state,

“³” in the expression “³n-π*” indicates a triplet state, and “¹” in the expressions “¹n-π*” and “¹π-π*” indicates a singlet state, and

the energy level in the ¹n-π* excited state, the energy level in the ¹π-π* excited state, and the energy level in the ³n-π* excited state may each independently calculated by using a time dependent-Density Functional Theory method (for example, a time dependent-Density Functional Theory method of a Gaussian 09 program) that is structurally optimized at a level of CAM-B3LYP/6-311+G(d,p).

Another aspect provides a fluorescent compound,
wherein the fluorescent compound has ³n-π*-to-¹π-π* energy transition from a ³n-π* excited state to a ¹π-π* excited state,
an energy level in a n-π* excited state of the fluorescent compound is greater than an energy level in the ¹π-π* excited state of the fluorescent compound,
the fluorescent compound emits a fluorescent light by radiative energy transition of an exciton in the ¹π-π* excited state to a ground state,

“³” in the expression “³n-π*” indicates a triplet state, and “¹” in the expressions “¹n-π*” and “¹π-π*” indicates a singlet state, and
the energy level in the ¹n-π* excited state, the energy level in the ¹π-π* excited state, and the energy level in the ³n-π* excited state are each independently calculated by using a time dependent-Density Functional Theory method (for example, a time dependent-Density Functional Theory method of a Gaussian 09 program) that is structurally optimized at a level of CAM-B3LYP/6-311+G(d,p).

According to an aspect, there is provided a fluorescent compound, wherein the fluorescent compound is not a coumarin-based compound represented by Formula 1′:

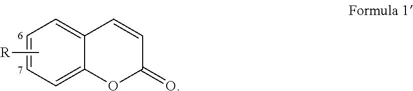

In Formula 1′, R may be a C₆-C₅₀ aryl group positioned at the 6-position or 7-position of a coumarin ring in Formula 1′.

### BRIEF DESCRIPTION OF THE DRAWINGS

These and/or other aspects will become apparent and more readily appreciated from the following description of the embodiments, taken in conjunction with the accompanying drawings in which:

FIG. 1 is an energy level diagram describing a luminescence mechanism of a fluorescent compound according to an embodiment;

FIG. 2 is an energy level diagram describing a luminescence mechanism of a fluorescent compound according to another embodiment;

FIG. 3 is a schematic cross-sectional view showing an organic light-emitting device according to an embodiment;

FIGS. 4 to 7 are each a graph of normalized photoluminescence intensity versus time (nanoseconds, ns) showing time-resolved PL spectroscopy (TRPL) plots with respect to Compound 4, BDpyInCz, Ir—C, and ACR, respectively;

FIG. 8A is a graph of absorption variation (ΔmOD, a.u.) versus wavelength (nanometer, nm) showing transient absorption spectra obtained for an N₂-saturated acetonitrile solution of Compound 4 when measured with respect to time at 0.001 ns, 0.01 ns, 0.1 ns, and 1 ns after photoexcitation, respectively;

FIG. 8B is a graph of absorption variation (ΔmOD, a.u.) versus time (picosecond, ps) and shows a decay trace and nonlinear least square fitting of an N₂-saturated acetonitrile solution of Compound 4 with respect to time for light having a wavelength of 1030 nm;

FIG. 9A is a graph of absorption variation (ΔmOD, a.u.) versus wavelength (nm) showing transient absorption spectra obtained for an air-saturated acetonitrile solution of Compound 4 when measured with respect to time at 0.001 ns, 0.01 ns, 0.1 ns, and 1 ns after photoexcitation, respectively; and

FIG. 9B is a graph of absorption variation (ΔmOD, a.u.) versus time (ps) and shows a decay trace and nonlinear least square fitting of an air-saturated acetonitrile solution of Compound 4 with respect to time for light having a wavelength of 1030 nm, respectively.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are described below, by referring to the figures, to explain certain aspects of the present description. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, the term “energy level of a ¹π-π* excited state” means the energy level of a singlet excited state obtained by excitation from a π-conjugate orbital to a π-conjugate orbital.

As used herein, the term “energy level of a ¹n-π*l excited state” means the energy level of a singlet excited state obtained by excitation from an isolated orbital to a π-conjugate orbital.

As used herein, the term “³n-π* excited state” means the energy level of a triplet excited state obtained by excitation from an isolated orbital to a π-conjugate orbital.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

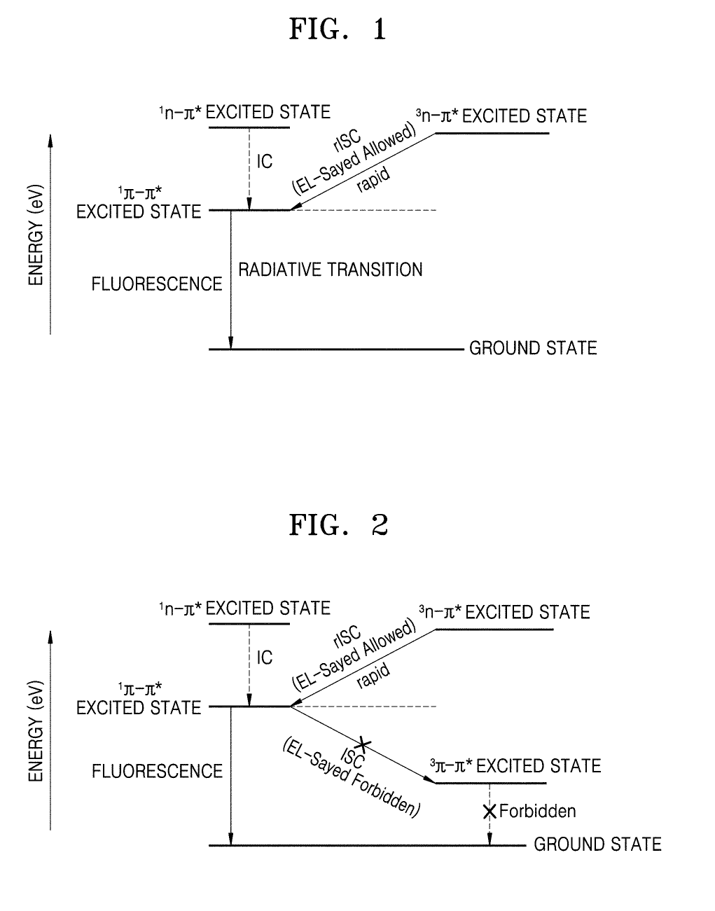

FIG. 1 is a diagram describing a luminescence mechanism of a fluorescent compound according to an embodiment. Hereinafter, a fluorescent compound will be described with reference to FIG. 1.

The fluorescent compound may have ³n-π*-to-¹π-π* energy transition from a ³n-π* excited state to a ¹π-π* excited state (hereinafter, “the ³n-π*-to-¹π-π* energy transition”). That is, the fluorescent compound may have a ³n-π* excited state, and the transition from the ³n-π* excited state to the ¹π-π* excited state may be measured.

In the fluorescent compound, the ³n-π*-to-¹π-π* energy transition may be confirmed by using an infrared (IR) transient absorption spectrometer (see Evaluation Example 3).

As shown in FIG. 1, an energy level in a ¹n-π* excited state of the fluorescent compound may be greater (or, higher) than an energy level in the ¹π-π* excited state of the fluorescent compound. The energy levels can be expressed in electron Volts (eV).

That is, an exciton in the ¹n-π* excited state may be non-radiatively transferred to the ¹π-π* excited state via internal conversion (IC), and does not contribute to light emission by direct transition to a ground state. Instead, a fluorescence may be emitted by radiative energy transition of an exciton in the ¹π-π* excited state of the fluorescent compound to a ground state of the fluorescent compound.

The energy level (eV) in the ¹π-π* excited state of the fluorescent compound may have two or more different values (see, e.g., Compound 4 in Table 1). In other words, the energy level in the ¹π-π* excited state can include one or more different energy levels in the ¹π-π* excited state.

In an embodiment, the energy level in the ³n-π* excited state of the fluorescent compound may be greater (or, higher) than the energy level in the ¹π-π* excited state of the fluorescent compound (see FIG. 1).

In one or more embodiments, the energy level in the ³n-π* excited state excited state of the fluorescent compound may be less (or, lower) than the energy level in the ¹π-π* excited state of the fluorescent compound.

That is, the fluorescent compound may satisfy the energy relationships wherein the energy level in the ¹n-π* excited state >the energy level in the ³n-π* excited state>the energy level in the ¹π-π* excited state, or wherein the energy level in the ¹n-π* excited state >the energy level in the ¹π-π* excited state >the energy level in the ³n-π* excited state.

In one or more embodiments, a difference between i) the energy level in the ³n-π* excited state of the fluorescent compound and ii) the lowest energy level of the one or more energy levels in the ¹π-π* excited state may be about 1 eV or less, for example, about 0.69 eV or less, and for example, about 0.3 eV or less. However, embodiments of the present disclosure are not limited thereto.

An exciton in the ³n-π* excited state of the fluorescent compound may be transferred to the ¹π-π* excited state via reverse intersystem crossing (rISC). The fluorescence (i.e., fluorescent light) may be emitted by radiative transition of the exciton in the ¹π-π* excited state, which was previously transferred from the ³n-π* excited state of the fluorescent compound to the ¹π-π* excited state of the fluorescent compound via reverse intersystem crossing (rISC), to a ground state of the fluorescent compound. By this, although the fluorescent compound is not an expensive transition metal-containing compound, for example, using iridium, platinum, or the like, the fluorescent compound may achieve a theoretical 100% emission efficiency upon harvesting of triplet excitons. The fluorescent compound may be clearly distinguished from a phosphorescent compound in the art in terms of not including a transition metal, such as iridium, platinum, or the like.

Here, the rISC may be an allowed crossing according to the EL-Sayed's Rule, and thus, may have a very rapid speed. For example, a rate of the rISC may be in a range of about 10⁶ inverse seconds (s⁻¹) to about 10⁸ s⁻¹. For example, the rate of the rISC may be in a range of about 10⁶ s⁻¹ to about 10⁷ s⁻¹, and in an embodiment, may be in a range of about 10⁷ s⁻¹ to about 10⁸ s⁻¹, but embodiments of the present disclosure are not limited thereto. The rate of the rISC may be measured according to time-resolved PL spectroscopy (TRPL).

Although not particularly limited to a specific theory, the exciton in the ³n-π* excited state of the fluorescent compound may be transferred to the ¹π-π* excited state via “the allowed and rapid” rISC according to the EL-Sayed's Rule, and thus, the fluorescent compound may have a relatively short exciton lifetime, i.e., a short decay time. For example, the fluorescent compound may have an exciton lifespan in a range of about 0.1 nanoseconds (ns) to about 1 microsecond (μs), in an embodiment, in a range of about 1 ns to about 0.1 μs, and in another embodiment, in a range of about 1 ns to about 0.01 μs. In this regard, the fluorescent compound may be clearly distinguished from a thermally activated delayed fluorescent (TADF) compound whose exciton lifespan is relatively long due to intramolecular charge transfer (ICT) which is “forbidden and relatively slow” rISC.

As described above, the fluorescent compound having a short exciton lifetime (or decay time) may have a lower probability of deterioration, compared with a TADF compound having a relatively long exciton lifetime. Thus, an electronic device, for example, an organic light-emitting device, including the fluorescent compound may have a longer lifetime.

In summary, the fluorescent compound having the emission mechanism of FIG. 1 may have the following advantages:
i) the fluorescent compound may emit fluorescence of high luminance and/or high emission efficiency, by radiative energy transition of an exciton thereof in the ¹π-π* excited state having a large oscillator intensity to a ground state; and
ii) the exciton of the fluorescent compound in the ¹π-π* excited state, which is “rapidly” transferred from the ³n-π* excited state of the fluorescent compound to the ¹π-π* excited state of the fluorescent compound via “allowed and rapid” reverse intersystem crossing (rISC) according to the EL-Sayed's Rule, may be also subjected to radiative energy transition from the ¹π-π* excited state to a ground state, and thus, the fluorescent compound may have a relatively short exciton lifetime (or decay time). Therefore, the fluorescent compound may have a minimized probability of deterioration.

In an embodiment, the energy level in the ³n-π* excited state of the fluorescent compound may be less (or, lower) than the energy level in the ³π-π* excited state of the fluorescent compound (for example, see Compound 4 in Table 1). In one or more embodiments, as shown in FIG. 2, the energy level in the ³n-π* excited state of the fluorescent compound may be greater (or, higher) than the energy level in the ³π-π* excited state in the fluorescent compound.

FIG. 2 is an energy diagram describing an emission mechanism of a fluorescent compound according to another embodiment.

The fluorescent compound of FIG. 2 may have the same emission mechanism as the fluorescent compound of FIG. 1, except that the energy level in the ³n-π* excited state may be greater (or, higher) than the energy level in the ³π-π* excited state. Here, the transfer of the exciton in the ¹π-π* excited state of the fluorescent compound to the ³π-π* excited state of the fluorescent compound via ISO is forbidden, and thus, substantially, the exciton in the ³n-π* excited state and the exciton in the ³π-π* excited state may not be subjected to directly-radiative transition to a ground state.

In the fluorescent compound, the energy level in the ³π-π* excited state may have two or more different values, that is, there can be a plurality of energy levels (or sublevels) in the energy level in the ³π-π* excited state.

In the present specification, “³” in the expressions “³n-π*” and “³π-π*” indicates a triplet state, and “¹” in the expressions “'n-π*” and “¹π-π*” indicates a singlet state. That is, the expressions “³n-π*” and “³π-π*” may be also respectively represented by “triplet n-π*” and “triplet π-π*”, and the expressions “'n-π*” and “¹π-π*” may be also respectively represented by “singlet n-π*” and “singlet π-π*”.

In the present specification, the energy level in the ¹n-π* excited state, the energy level in the ¹π-π* excited state, the energy level in the ³n-π* excited state, and the energy level in the ³π-π* excited state may each independently be calculated by using the structurally optimized time dependent-Density Functional Theory (TD-DFT) at a level of CAM-B3LYP/6-3 1 1+G(d,p), for example, using the Gaussian 09 program. A detailed description for the calculation may be referred by Evaluation Examples below.

The fluorescent compound may include a non-bonding orbital (for example, a non-bonding π orbital) which is able to induce the above-described ³n-π*-to-¹π-π* energy transition.

For example, the fluorescent compound may include at least one carbonyl group.

In an embodiment, the fluorescent compound may be represented by Formula 1 or 2.

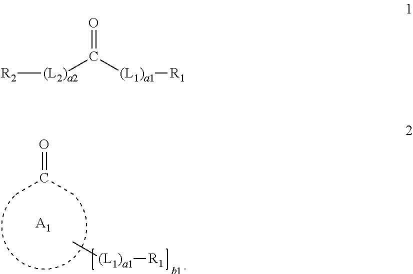

In Formulae 1 and 2,
ring A₁ may be a carbonyl-containing C₅-C₆₀ carbocyclic group or a carbonyl-containing C₁-C₆₀ heterocyclic group,
each L₁ and L2 may be the same or different, and each independently may be a substituted or unsubstituted C₁-C₆₀ alkylene group, a substituted or unsubstituted C₂-C₆₀ alkenylene group, a substituted or unsubstituted C₂-C₆₀ alkynylene group, a substituted or unsubstituted C₃-C₁₀ cycloalkylene group, a substituted or unsubstituted heterocycloalkylene group, a substituted or unsubstituted C₃-C₁₀ cycloalkenylene group, a substituted or unsubstituted heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group,
a1 and a2 may each independently be an integer from 0 to 20,

R₁ and R₂ may each independently be hydrogen, deuterium, —F, —Cl, —Br, —I, —SFS, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), or —P(═O)(Q₈)(Q₉),
b1 may be an integer from 1 to 20,
at least one substituent of the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, substituted divalent non-aromatic condensed polycyclic group, substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), or —P(═O)(Q₁₈)(Q₁₉);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆o heteroaryl group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), or —P(═O)(Q₂₈)(Q₂₉); or

—N(Q₃₁)(Q₃₂), —Si(Q₃₃)(Q₃₄)(Q₃₅), —B(Q₃₆)(Q₃₇), or —P(═O)(Q₃₈)(Q₃₉), and

Q₁ to Q₉, Q₁₁ to Q₁₉, Q₂₁ to Q₂₉, and Q₃₁ to Q₃₉ may each independently be hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkyl group substituted with at least one of deuterium, a C₁-C₆₀ alkyl group, or a C₆-C₆₀ aryl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one of deuterium, a C₁-C₆₀ alkyl group, and a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group.

For example, ring A₁ in Formula 2 may be i) a C₂-C₅ 5-membered ring including a carbonyl group, ii) a C₂-C₆ 6-membered ring including a carbonyl group, or iii) a condensed ring in which at least one first ring and at least one second ring are condensed, wherein the first ring is a C₂-C₅ 5-membered ring including a carbonyl group or a C₂-C_{6 6}-membered ring including a carbonyl group, and the second ring is a C₂-C₅ 5-membered ring including a carbonyl group, a C₂-C₆ 6-membered ring including a carbonyl group, a C₂-C₅ 5-membered ring not including a carbonyl group, or a C₂-C₆ 6-membered ring not including a carbonyl group.

The terms “a carbonyl-containing Cs-Coo carbocyclic group”, “a carbonyl-containing C₁-C₆₀ heterocyclic group”, “a C₂-C₅ 5-membered ring including a carbonyl group” and “a C₂-C_{6 6}-membered ring including a carbonyl group” may each be a cyclic group including at least one carbonyl group as a ring-forming moiety, and the terms “a C₂-C_{5 5}-membered ring not including a carbonyl group” and “a C₂-C_{6 6}-membered ring not including a carbonyl group” may each be a cyclic group not including a carbonyl group as a ring-forming moiety.

In the specification, the carbon of the carbonyl group included as a ring-forming moiety in “a carbonyl-containing C₅-C₆₀ carbocyclic group”, “a carbonyl-containing C₁-C₆₀ heterocyclic group”, “a C₂-C₅ 5-membered ring including a carbonyl group”, and “a C₂-C₆ 6-membered ring including a carbonyl group” is also counted as a ring-forming atom. Thus, for example, a C₂-C₅ 5-membered ring including a carbonyl group is a cyclic group having a carbon atom of the carbonyl group and 1 to 4 other carbon atoms as ring members.

In an embodiment, the ring A₁ may include at least one carbon as a ring-forming atom, or at least one carbon and at least one oxygen as a ring-forming atom.

In another embodiment, ring-forming atoms of the ring A₁ may be carbon, or atoms of the ring A₁ may consist of carbon and oxygen.

In other embodiment, a core represented by the formula

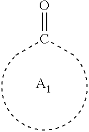

in Formula 2 may be a group represented by one of Formulae 2-1 to 2-15, but embodiments of the present disclosure are not limited thereto:

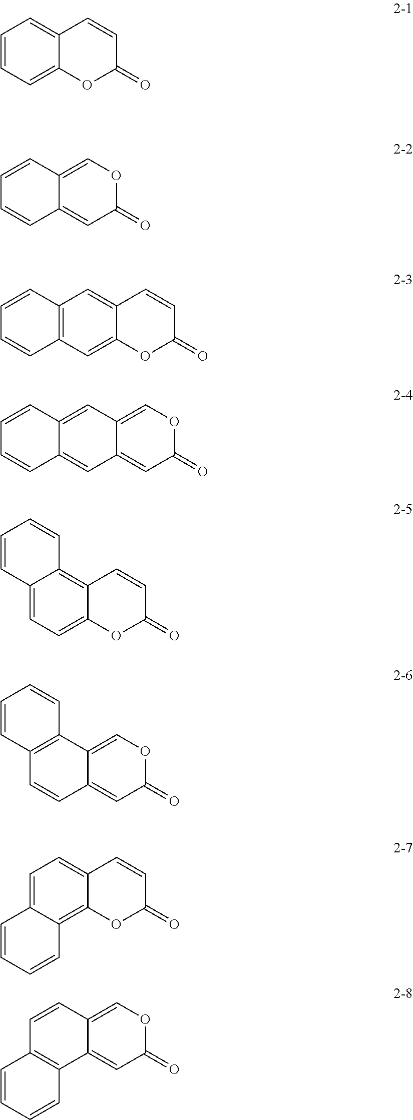

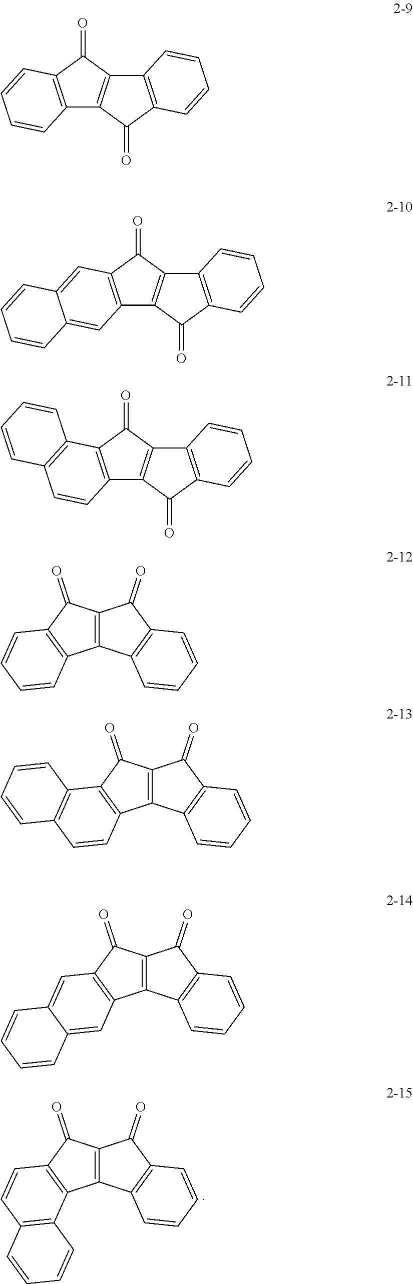

For example, a group represented by Formula 2-1 may be interpreted as a condensed ring in which a first ring, which is a C₅ 6-membered ring including a carbonyl group, and a second ring, which is a C₆ 6-membered ring not including a carbonyl group (such as a phenylene group), are condensed.

In an embodiment, a group represented by Formula 2-9 may be interpreted as a condensed ring in which two first rings, each of which is a C₅ 5-membered ring including a carbonyl group, and two second rings, each of which is a C₆ 6-membered ring not including a carbonyl group (such as a phenylene group), are condensed.

In one or more embodiments, in Formulae 1 and 2, each L₁ and L₂ may be the same or different, and may each independently be a C₁-C₂₀ alkylene group, a C₂-C₂₀ alkenylene group, a C₂-C₂₀ alkynylene group, a phenylene group, a pentalenylene group, an indenylene group, a naphthylene group, an azulenylene group, a heptalenylene group, an indacenylene group, an acenaphthylene group, a fluorenylene group, a spiro-bifluorenylene group, a benzofluorenylene group, a dibenzofluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, a pentaphenylene group, a hexacenylene group, a pentacenylene group, a rubicenylene group, a coronenylene group, an ovalenylene group, a pyrrolylene group, a thiophenylene group, a furanylene group, an imidazolylene group, a pyrazolylene group, a thiazolylene group, an isothiazolylene group, an oxazolylene group, an isoxazolylene group, a pyridinylene group, a pyrazinylene group, a pyrimidinylene group, a pyridazinylene group, an isoindolylene group, an indolylene group, an indazolylene group, a purinylene group, a quinolinylene group, an isoquinolinylene group, a benzoquinolinylene group, a phthalazinylene group, a naphthyridinylene group, a quinoxalinylene group, a quinazolinylene group, a cinnolinylene group, a carbazolylene group, a phenanthridinylene group, an acridinylene group, a phenanthrolinylene group, a phenazinylene group, a benzimidazolylene group, a benzofuranylene group, a benzothiophenylene group, an isobenzothiazolylene group, a benzoxazolylene group, an isobenzoxazolylene group, a triazolylene group, a tetrazolylene group, an oxadiazolylene group, a triazinylene group, a dibenzofuranylene group, a dibenzothiophenylene group, a benzocarbazolylene group, a dibenzocarbazolylene group, or an imidazopyridinylene group, each unsubstituted or substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, or —Si(Q₃₃)(Q₃₄)(Q₃₅). Here, Q₃₃ to Q₃₅ may each independently be the same as described above.

In one or more embodiments, in Formulae 1 and 2, a1 and a2 each indicate the number of L₁ units and the number of L₂ units, wherein, when a1 is 0, *—(L₁)ₐ₁—*¹ may be a single bond, and when a2 is 0, *—(L₂)ₐ₂—*¹ may be a single bond. When a1 is two or more, two or more of L₁ units may be identical to or different from each other, and when a2 is two or more, two or more of L₂ units may be identical to or different from each other.

For example, in Formulae 1 and 2, al and a2 may each independently be 0, 1, 2, or 3, but embodiments of the present disclosure are not limited thereto.

In one or more embodiments, R₁ and R₂ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, C₁-C₂₀ alkyl group, or a C₁-C₂₀ alkoxy group;
a C₁-₂₀ alkyl group and a C₁-C₂₀ alkoxy group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a pyridinyl group, or a pyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, and an imidazopyrimidinyl group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a cyclopentyl group, a cyclohexyl group, a cycloheptyl group, a cycloctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a dibenzosilolyl group, a benzocarbazolyl group, a dibenzocarbazolyl group, an imidazopyridinyl group, an imidazopyrimidinyl group, or —Si(Q₃₃)(Q₃₄)(Q₃₅); or
—N(Q₁)(Q₂), —Si(Q₃)(Q₄)(Q₅), —B(Q₆)(Q₇), or —P(═O)(Q₈)(Q₉), and
Q₁ to Q₉ and Q₃₃ to Q₃₅ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one of deuterium, a C₁-C₁₀ alkyl group, or a phenyl group,
but embodiments of the present disclosure are not limited thereto.

In Formula 2, b1 indicates the number of *—(L₁)ₐ₁—R₁ moieties, wherein, when b1 is two or more, two or more of *—(L₁)ₐ₁—R₁(s) moieties may be identical to or different from each other.

For example, b1 may be 1, 2, 3, or 4, but embodiments of the present disclosure are not limited thereto.

In one or more embodiments, the fluorescent compound may be one of Compounds 1 to 8, but embodiments of the present disclosure are not limited thereto:

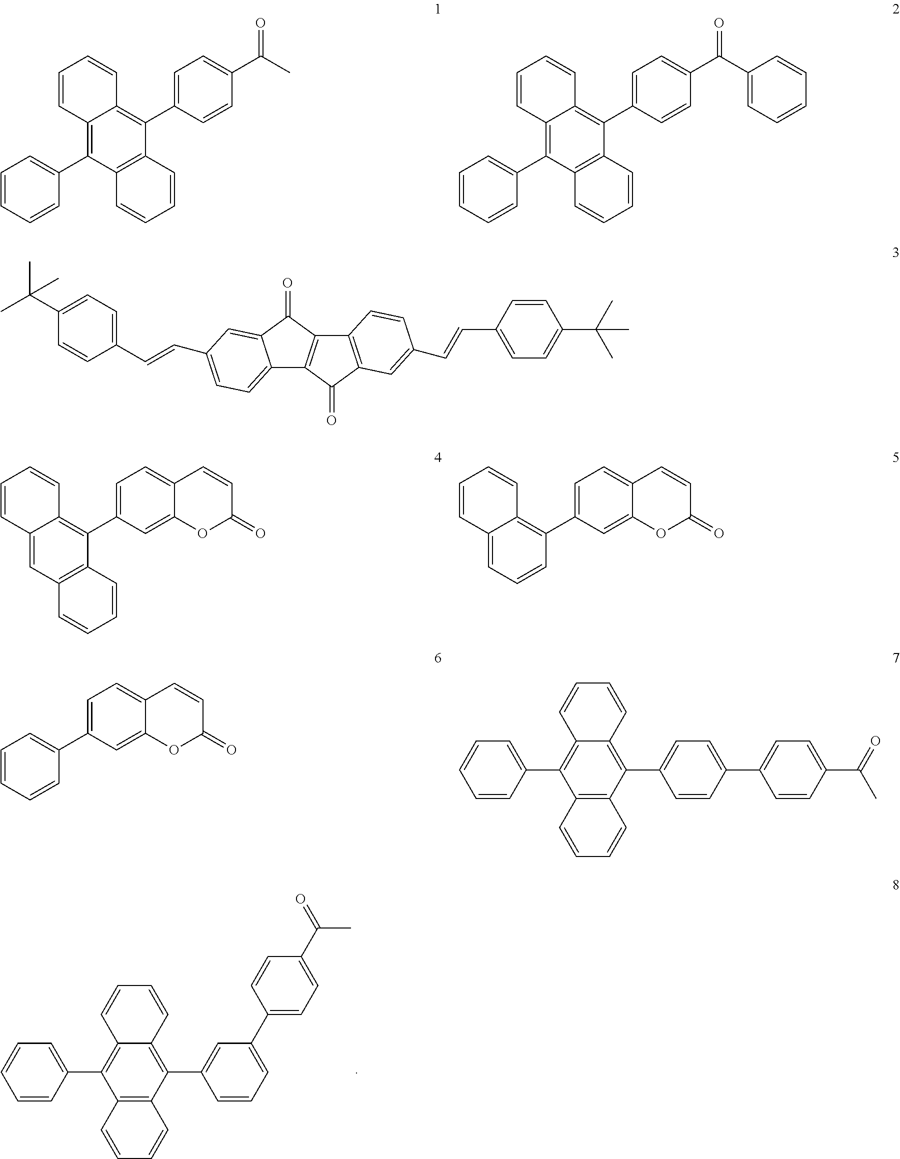

In an embodiment, the above-described fluorescent compound may be included in an emission layer of an organic light-emitting device.

The fluorescent compound included in the emission layer may emit light, for example, emit a fluorescent light according to the emission mechanism of FIG. 1 or 2.

Thus, in an embodiment, a ratio of an emission portion of the fluorescence emitted by radiative energy transition of the exciton thereof in the ¹π-π* excited state to a ground stand, where the exciton is previously transferred from the ³n-π* excited state of the fluorescent compound to the ¹π-π* excited state of the fluorescent compound via reverse intersystem crossing (rISC), to a total emission portion emitted from the emission layer may be at least 90%, for example, at least 92%, and in another embodiment, at least 95%. However, embodiments of the present disclosure are not limited thereto. In other words, at least 90% of the total light emission from the emission layer is the fluorescent light emission from the fluorescent compound via reverse intersystem crossing.

In an embodiment, the emission layer may consist essentially of, or consist of, the fluorescent compound.

In one or more embodiments, the emission layer may further include, in addition to the above-described fluorescent compound, a host. The host may be one compound, or a combination of two or more compounds. When the emission layer further includes a host, the fluorescent compound included in the emission layer may serve as a fluorescence emitter, wherein an amount of the fluorescent compound may be smaller than that of the amount of the host in the emission layer.

For example, the host may include at least one of a fluorene-containing compound, a carbazole-containing compound, a dibenzofuran-containing compound, a dibenzothiophene-containing compound, an indeno carbazole-containing compound, an indolocarbazole-containing compound, a benzofurocarbazole-containing compound, a benzothienocarbazole-containing compound, an acridine-containing compound, a dihydroacridine-containing compound, a triindolobenzene-containing compound, a pyridine-containing compound, a pyrimidine-containing compound, a triazine-containing compound, a silicon-containing compound, a cyano group-containing compound, a phosphine oxide-containing compound, a sulfoxide-containing compound, or a sulfonyl-containing compound.

For example, the host may be a compound including at least one carbazole ring and at least one cyano group, or a phosphine oxide-containing compound, but embodiments of the present disclosure are not limited thereto.

The host may include, for example, at least one compound that is 4,4-N,N′-dicarbazole-1,1′-biphenyl (CBP), mCBP (Compound H7 below), or Compounds H1 to H24 below, but embodiments of the present disclosure are not limited thereto:

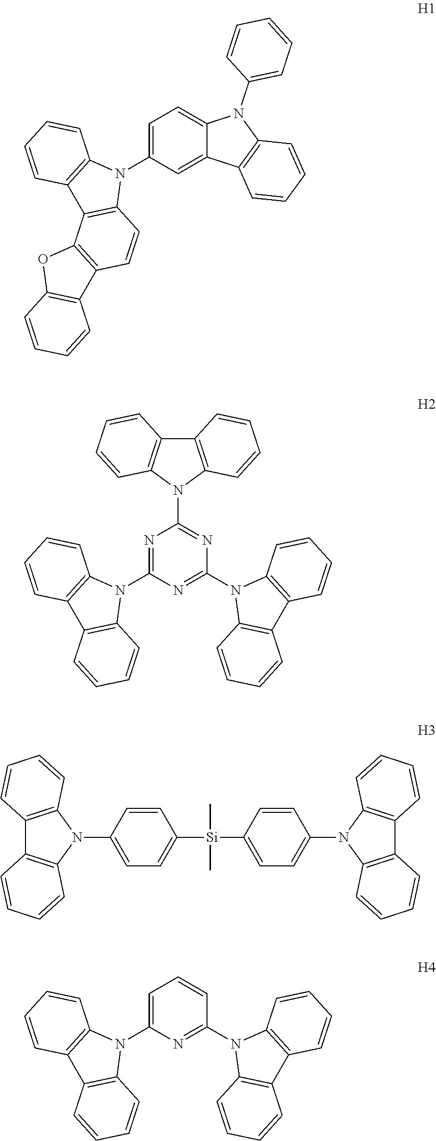 [SMILES: c1ccc(-n2c3ccccc3c3cc(-n4c5ccccc5c5c6oc7ccccc7c6ccc54)ccc32)cc1 ; c1ccc2c(c1)c1ccccc1n2-c1nc(-n2c3ccccc3c3ccccc32)nc(-n2c3ccccc3c3ccccc32)n1 ; C[Si](C)(c1ccc(-n2c3ccccc3c3ccccc32)cc1)c1ccc(-n2c3ccccc3c3ccccc32)cc1 ; c1cc(-n2c3ccccc3c3ccccc32)nc(-n2c3ccccc3c3ccccc32)c1]

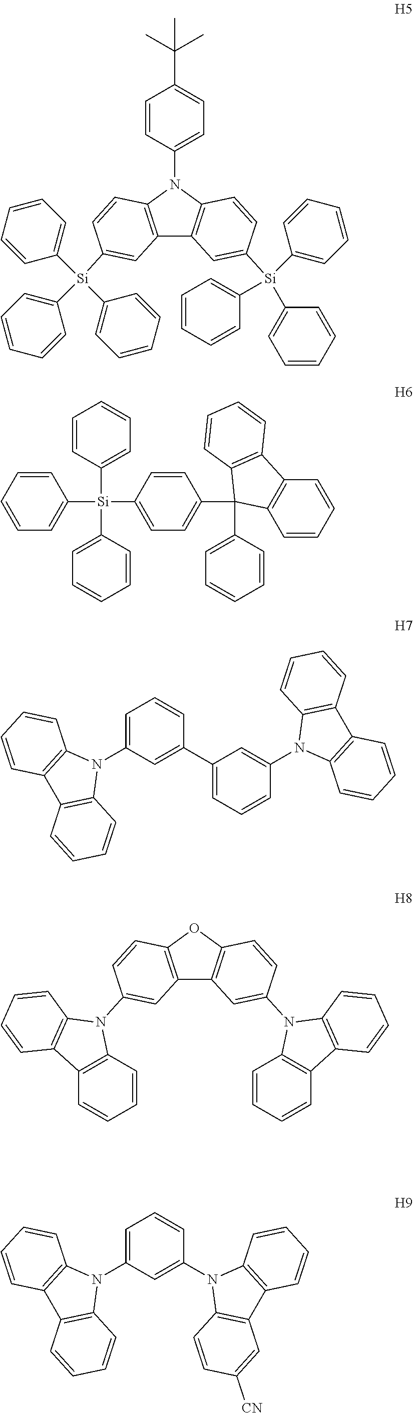 [SMILES: CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; c1ccc(C2(c3ccc([Si](c4ccccc4)(c4ccccc4)c4ccccc4)cc3)c3ccccc3-c3ccccc32)cc1 ; c1cc(-c2cccc(-n3c4ccccc4c4ccccc43)c2)cc(-n2c3ccccc3c3ccccc32)c1 ; c1ccc2c(c1)c1ccccc1n2-c1ccc2oc3ccc(-n4c5ccccc5c5ccccc54)cc3c2c1 ; N#Cc1ccc2c(c1)c1ccccc1n2-c1cccc(-n2c3ccccc3c3ccccc32)c1]

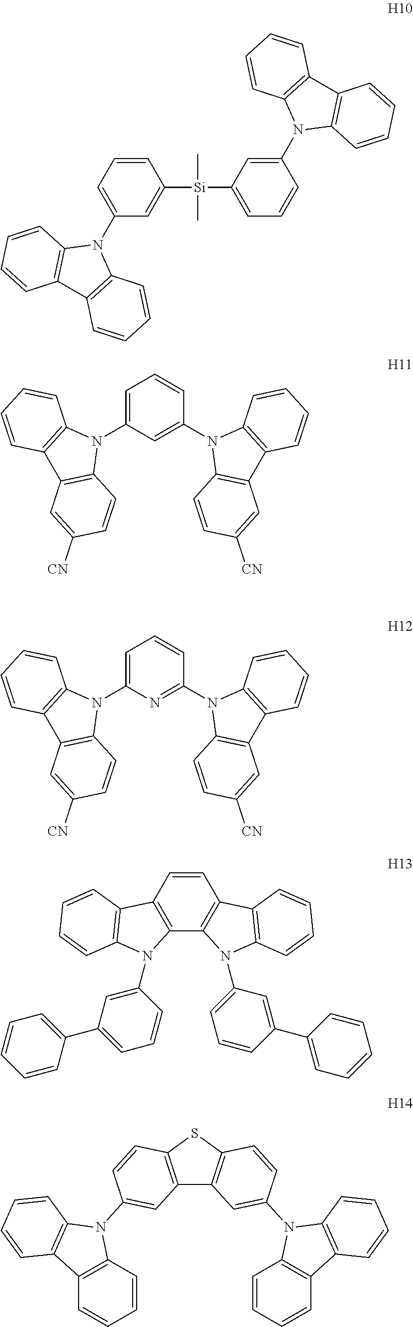

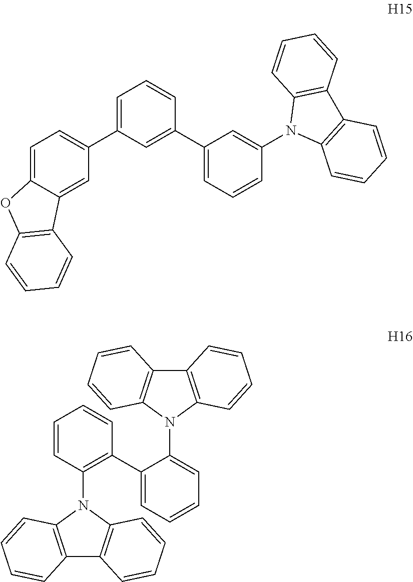 [SMILES: c1cc(-c2cccc(-n3c4ccccc4c4ccccc43)c2)cc(-c2ccc3oc4ccccc4c3c2)c1 ; c1ccc(-n2c3ccccc3c3ccccc32)c(-c2ccccc2-n2c3ccccc3c3ccccc32)c1]

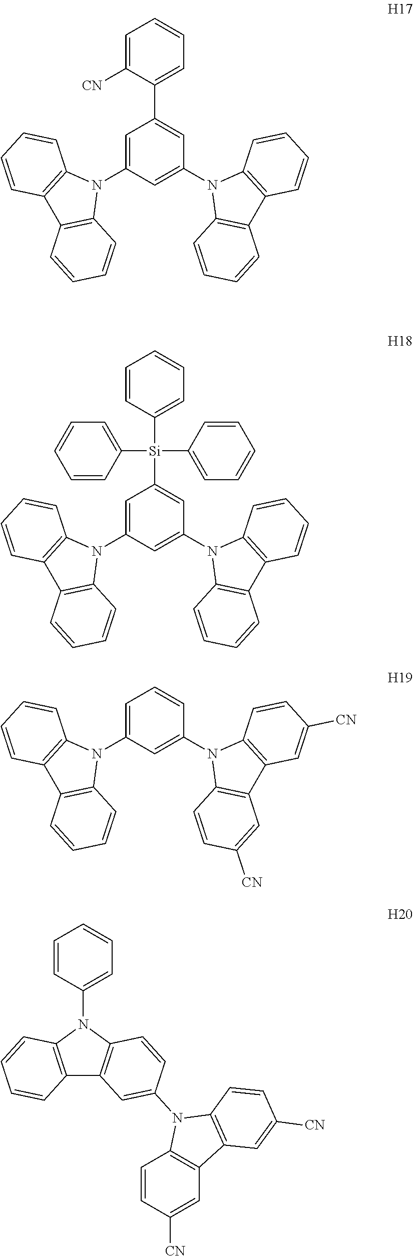

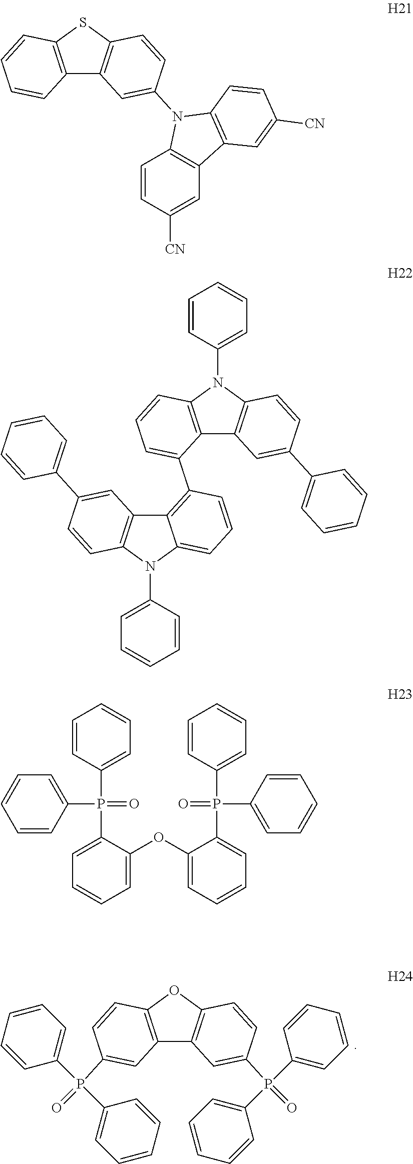 [SMILES: N#Cc1ccc2c(c1)c1cc(C#N)ccc1n2-c1ccc2sc3ccccc3c2c1 ; c1ccc(-c2ccc3c(c2)c2c(-c4cccc5c4c4cc(-c6ccccc6)ccc4n5-c4ccccc4)cccc2n3-c2ccccc2)cc1 ; O=P(c1ccccc1)(c1ccccc1)c1ccccc1Oc1ccccc1P(=O)(c1ccccc1)c1ccccc1 ; O=P(c1ccccc1)(c1ccccc1)c1ccc2oc3ccc(P(=O)(c4ccccc4)c4ccccc4)cc3c2c1]

The organic light-emitting device includes a hole transport region disposed between the first electrode and the emission layer and an electron transport region disposed between the emission layer and the second electrode, and the hole transport region includes a hole injection layer, a hole transport layer, an electron blocking layer, an buffer layer or a combination thereof, and the electron transport region includes a hole blocking layer, an electron transport layer, an electron injection layer, or a combination thereof.

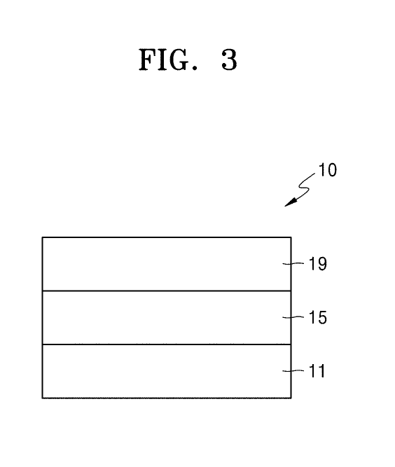

FIG. 3 is a schematic view of an organic light-emitting device 10 according to an embodiment. Hereinafter, the structure of an organic light-emitting device according to an embodiment and a method of manufacturing an organic light-emitting device according to an embodiment will be described in connection with FIG. 3. The organic light-emitting device 10 includes a first electrode 11, an organic layer 15, and a second electrode 19, which are sequentially stacked.

A substrate may be additionally disposed under the first electrode 11 or above the second electrode 19. For use as the substrate, any suitable substrate that is used in general organic light-emitting devices may be used, and the substrate may be a glass substrate or a transparent plastic substrate, each having excellent mechanical strength, thermal stability, transparency, surface smoothness, ease of handling, and water resistance.

The first electrode 11 may be formed by depositing or sputtering a material for forming the first electrode 11 on the substrate. The first electrode 11 may be an anode. The material for forming the first electrode 11 may be suitable materials with a high work function to facilitate hole injection. The first electrode 11 may be a reflective electrode, a semi-transmissive electrode, or a transmissive electrode. The material for forming the first electrode may be, for example, indium tin oxide (ITO), indium zinc oxide (IZO), tin oxide (SnO₂), and zinc oxide (ZnO). In one or more embodiments, magnesium (Mg), aluminum (Al), aluminum-lithium (Al—Li), calcium (Ca), magnesium-indium (Mg—In), or magnesium-silver (Mg—Ag) may be used as the material for forming the first electrode.

The first electrode 11 may have a single-layered structure or a multi-layered structure including two or more layers. For example, the first electrode 11 may have a three-layered structure of ITO/Ag/ITO, but the structure of the first electrode 11 is not limited thereto.

The organic layer 15 is disposed on the first electrode 11.

The organic layer 15 may include a hole transport region, an emission layer, and an electron transport region.

The hole transport region may be disposed between the first electrode 11 and the emission layer.

The hole transport region may include a hole injection layer, a hole transport layer, an electron blocking layer, a buffer layer, or a combination thereof.

The hole transport region may include a hole injection layer or a hole transport layer. In one or more embodiments, the hole transport region may have a hole injection layer/hole transport layer structure or a hole injection layer/hole transport layer/electron blocking layer structure, which are sequentially stacked in this stated order from the first electrode 11.

A hole injection layer may be formed on the first electrode 11 by using one or more suitable methods including vacuum deposition, spin coating, casting, or Langmuir-Blodgett (LB) deposition.

When a hole injection layer is formed by vacuum deposition, the deposition conditions may vary according to a compound that is used to form the hole injection layer, and the structure and thermal characteristics of the hole injection layer. For example, the deposition conditions may include a deposition temperature of about 100 to about 500° C., a vacuum pressure of about 10⁻⁸ to about 10⁻³ torr, and a deposition rate of about 0.01 Angstroms per second (Å/sec) to about 100 Å/sec. However, the deposition conditions are not limited thereto.

When the hole injection layer is formed using spin coating, coating conditions may vary according to the material used to form the hole injection layer, and the structure and thermal properties of the hole injection layer. For example, a coating speed may be from about 2,000 rpm to about 5,000 rpm, and a temperature at which a heat treatment is performed to remove a solvent after coating may be from about 80° C. to about 200° C. However, the coating conditions are not limited thereto.

Conditions for forming a hole transport layer and an electron blocking layer may be understood by referring to conditions for forming the hole injection layer.

The hole transport region may include at least one of m-MTDATA, TDATA, 2-TNATA, NPB, 13-NPB, TPD, Spiro-TPD, Spiro-NPB, methylated-NPB, TAPC, HMTPD, 4,4′,4″-tris(N-carbazolyl)triphenylamine (TCTA), polyaniline/dodecylbenzene sulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201 below, or a compound represented by Formula 202 below:

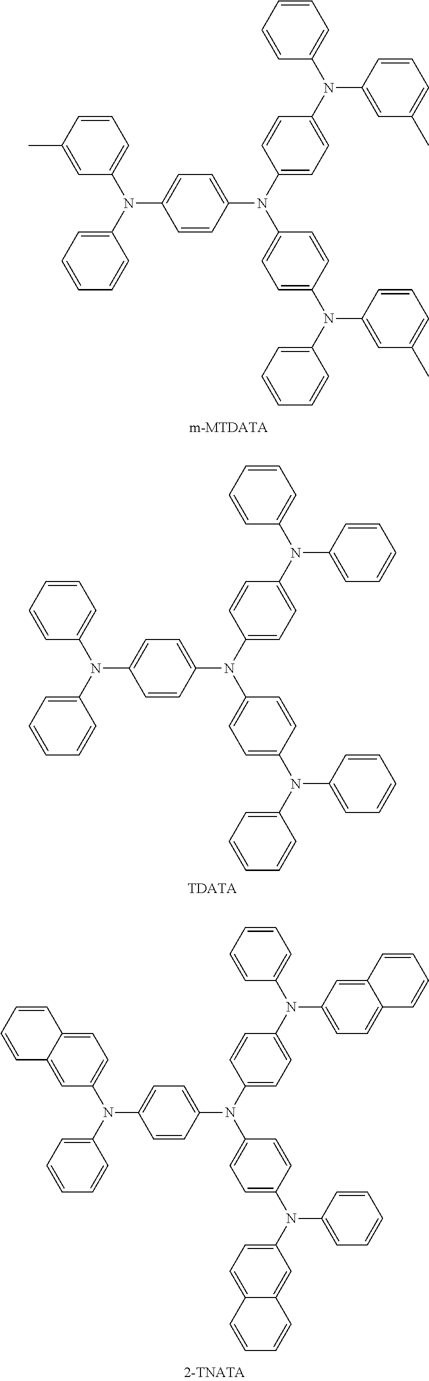

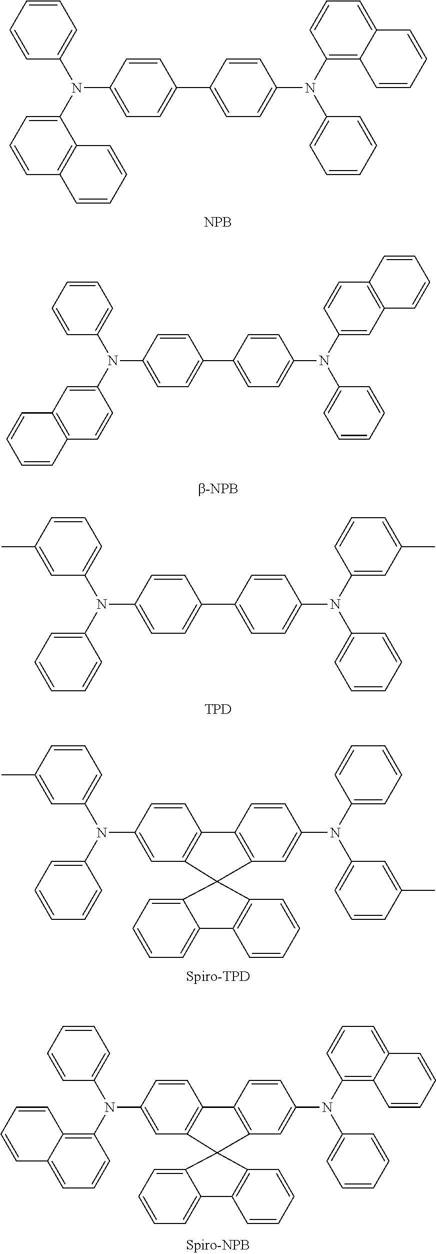

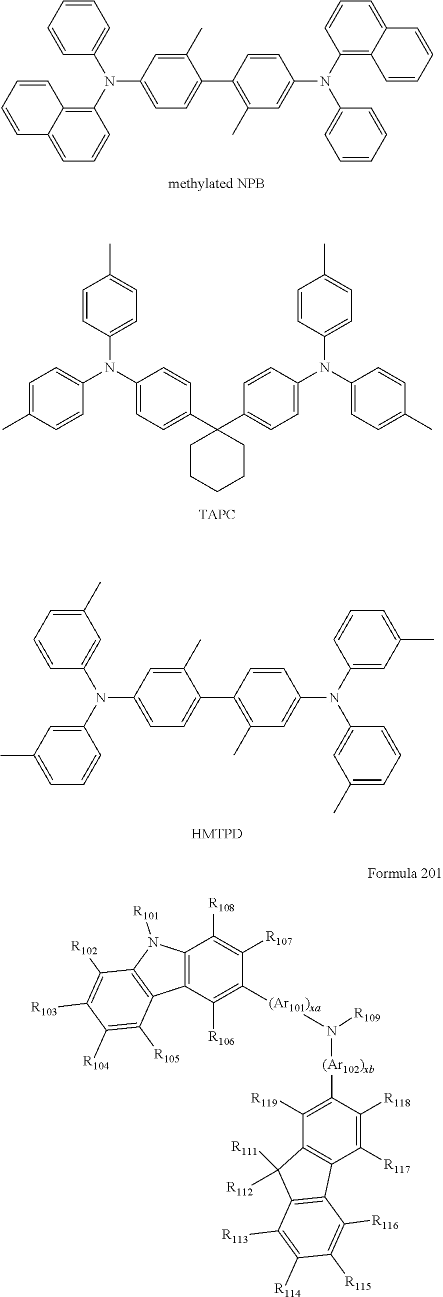

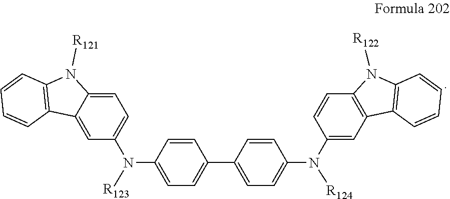

Ar₁₀₁ and Ar₁₀₂ in Formula 201 may each independently be:
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, or a pentacenylene group; or
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylenylene group, a naphthacenylene group, a picenylene group, a perylenylene group, and a pentacenylene group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arythio group, a C₁-C₆₀ heteroaryl group, a monovalent non-aromatic condensed polycyclic group, or a monovalent non-aromatic condensed heteropolycyclic group.

In Formula 201, xa and xb may each independently be an integer from 0 to 5, or may be 0, 1, or 2. For example, xa is 1 and xb is 0, but xa and xb are not limited thereto.

R₁₀₁ to R₁₀₈, R₁₁₁ to R₁₁₉, and R₁₂₁ to R₁₂₄ in Formulae 201 and 202 may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, a hexyl group, or the like), or a C₁-C₁₀ alkoxy group (for example, a methoxy group, an ethoxy group, a propoxy group, a butoxy group, a pentoxy group, or the like);
a C₁-C₁₀ alkyl group or a C₁-C₁₀ alkoxy group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a phenyl group, a naphthyl group, an anthracenyl group, a fluorenyl group, and a pyrenyl group; or
a phenyl group, a naphthyl group, an anthracenyl group, a fluorenyl group, or a pyrenyl group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, or a C₁-C₁₀ alkoxy group, but embodiments of the present disclosure are not limited thereto.

In Formula 201, R₁₀₉ may be:
a phenyl group, a naphthyl group, an anthracenyl group, and a pyridinyl group; and
a phenyl group, a naphthyl group, an anthracenyl group, or a pyridinyl group, each substituted with at least one of deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a phenyl group, a naphthyl group, an anthracenyl group, or a pyridinyl group.

In an embodiment, the compound represented by Formula 201 may be represented by Formula 201A, but embodiments of the present disclosure are not limited thereto:

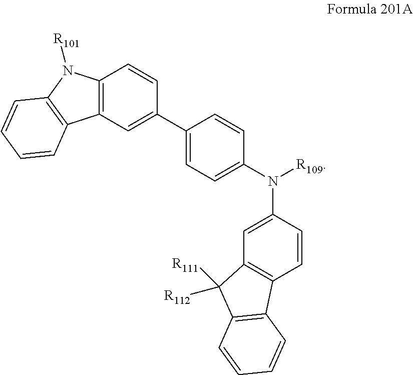

R₁₀₁, R₁₁₁, R₁₁₂, and R₁₀₉ in Formula 201A may be understood by referring to the description provided hereinabove.

For example, the compound represented by Formula 201, and the compound represented by Formula 202 may include compounds HT1 to HT20 illustrated below, but are not limited thereto:

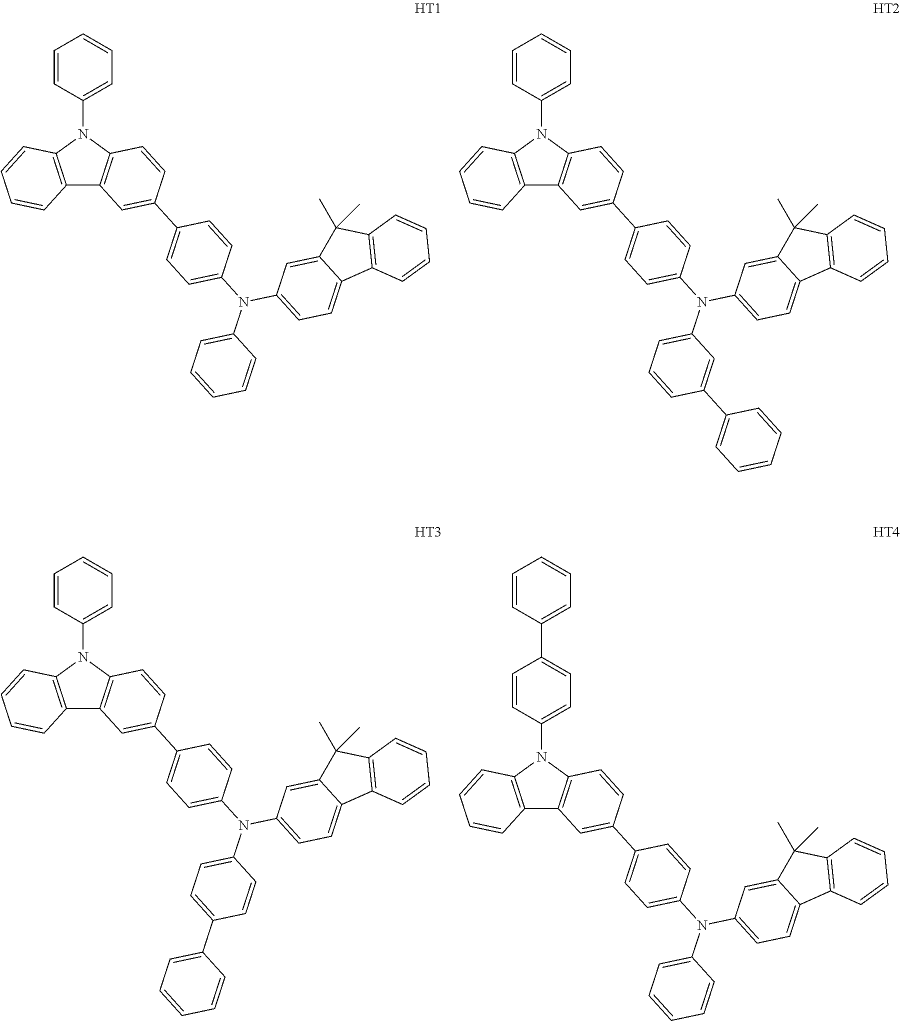

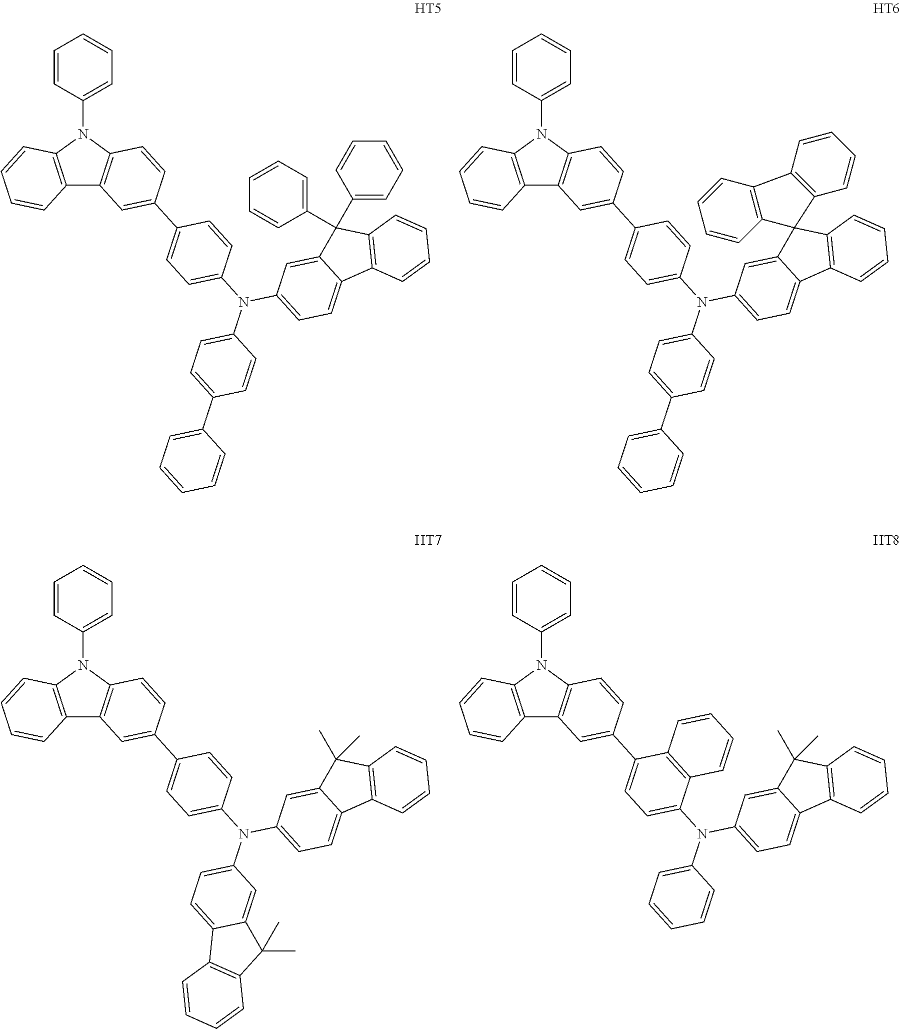 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccccc3)c3ccc(-c4ccc5c(c4)c4ccccc4n5-c4ccccc4)c4ccccc34)cc21]

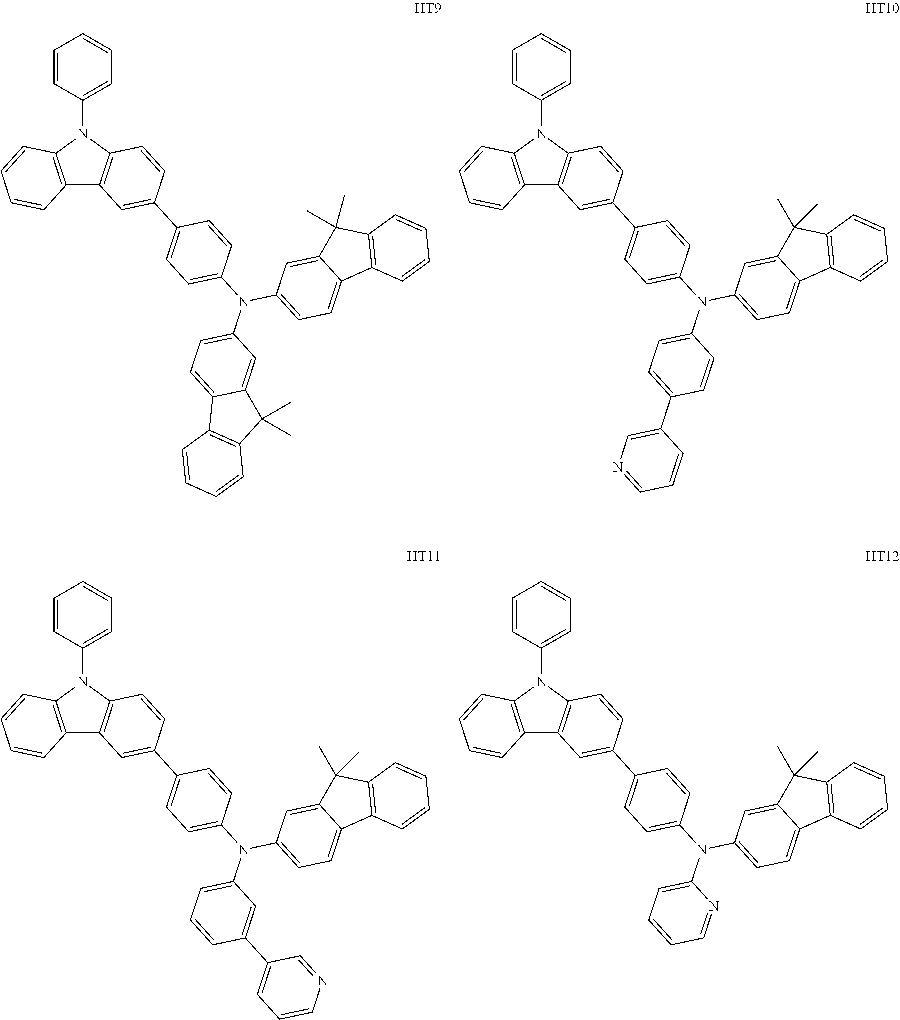 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21]

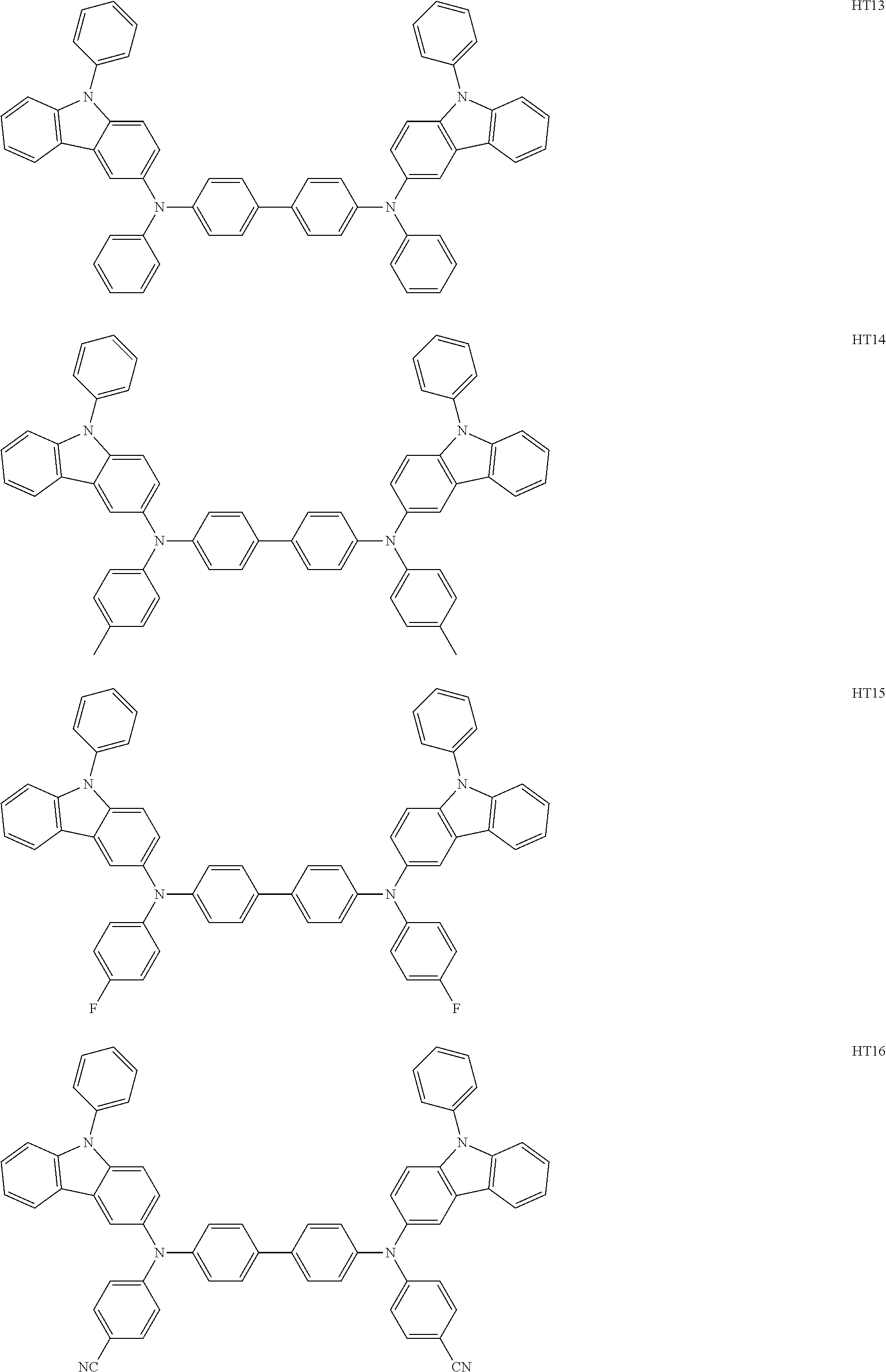

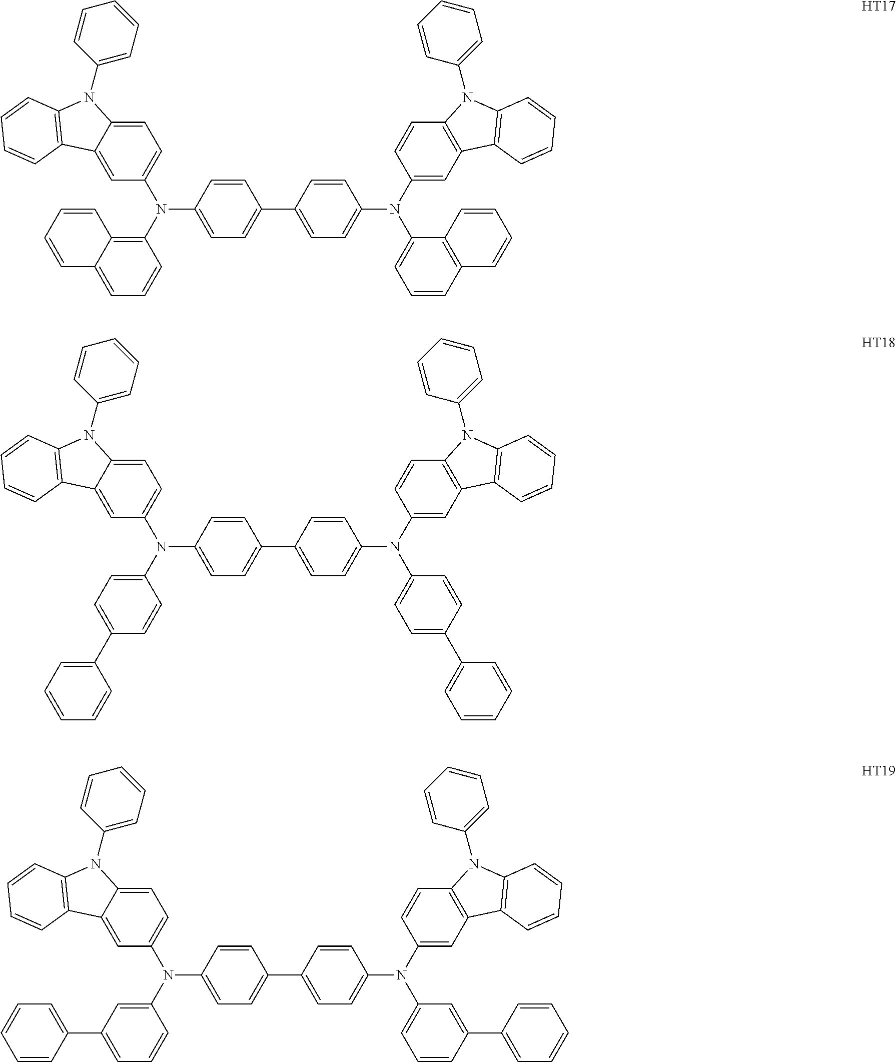

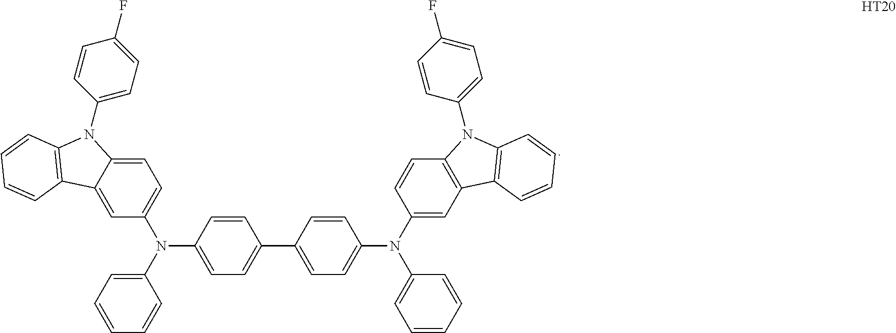 [SMILES: Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1 ; "HT20"]

A thickness of the hole transport region may be in a range of about 100 Å to about 10,000 Å, for example, about 100 Å to about 3,000 Å. When the hole transport region includes at least one of a hole injection layer and a hole transport layer, the thickness of the hole injection layer may be in a range of about 100 Å to about 10,000 Å, and for example, about 100 Å to about 2,000 Å, and the thickness of the hole transport layer may be in a range of about 50 Å to about 2,000 Å, and for example, about 100 Å to about 1500 Å. When the thicknesses of the hole transport region, the hole injection layer, and the hole transport layer are within these ranges, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one of a quinone derivative, a metal oxide, and a cyano group-containing compound, but embodiments of the present disclosure are not limited thereto. Non-limiting examples of the p-dopant are a quinone derivative, such as tetracyanoquinonedimethane (TCNQ) or 2,3,5,6-tetrafluoro-tetracyano-1,4-benzoquinonedimethane (F4-TCNQ); a metal oxide, such as a tungsten oxide or a molybdenum oxide; or a cyano group-containing compound, such as Compound HT-D1 or Compound HT-D2 below, but are not limited thereto:

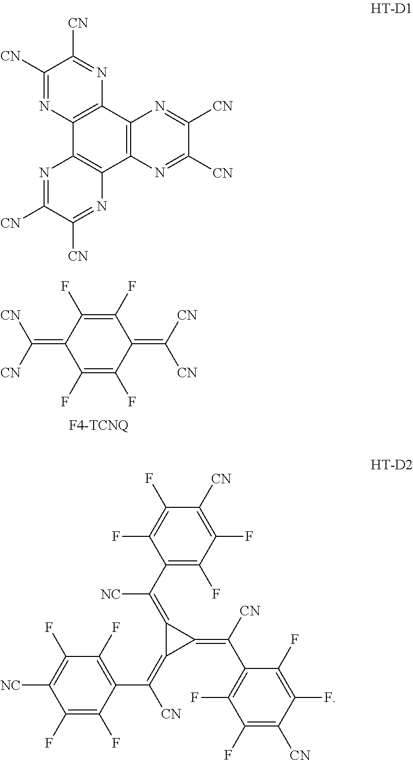

The hole transport region may include a buffer layer.

Also, the buffer layer may compensate for an optical resonance distance according to a wavelength of light emitted from the emission layer, and thus, efficiency of a formed organic light-emitting device may be improved.

The electron transport region may further include an electron blocking layer. The electron blocking layer may include, for example, mCP, but a material therefor is not limited thereto:

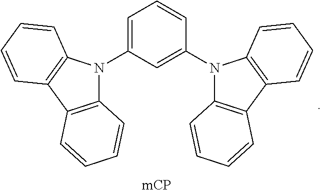 [SMILES: c1cc(-n2c3ccccc3c3ccccc32)cc(-n2c3ccccc3c3ccccc32)c1]

For example, as a material for the electron blocking layer, the host included in the emission layer may be used, but embodiments of the present disclosure are not limited thereto.

Then, an emission layer may be formed on the hole transport region by vacuum deposition, spin coating, casting, LB deposition, or the like. When the emission layer is formed by vacuum deposition or spin coating, the deposition or coating conditions may be similar to those applied in forming the hole injection layer although the deposition or coating conditions may vary according to a compound that is used to form the emission layer.

When the organic light-emitting device is a full-color organic light-emitting device, the emission layer may be patterned into a red emission layer, a green emission layer, and a blue emission layer. In one or more embodiments, due to a stacked structure including a red emission layer, a green emission layer, and/or a blue emission layer, the emission layer may emit white light.

The emission layer may include a fluorescent compound that satisfies the conditions described herein. The emission layer may include, consist essentially of, or consist of, the fluorescent compound, or may further include a host in addition to the fluorescent compound. The host is the same as described above.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

Then, an electron transport region may be disposed on the emission layer.

The electron transport region may include a hole blocking layer, an electron transport layer, an electron injection layer, or a combination thereof.

For example, the electron transport region may have a hole blocking layer/electron transport layer/electron injection layer structure or an electron transport layer/electron injection layer structure, but the structure of the electron transport region is not limited thereto. The electron transport layer may have a single-layered structure or a multi-layered structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP and Bphen, but may also include other materials:

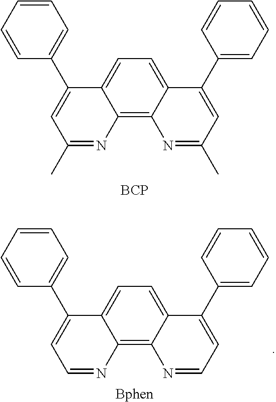

For example, as a material for the hole blocking layer, a compound identical to the host included in the emission layer may be used, but embodiments of the present disclosure are not limited thereto.

A thickness of the hole blocking layer may be in a range of about 20 Å to about 1,000 Å, for example, about 30 Å to about 300 Å. When the thickness of the hole blocking layer is within these ranges, the hole blocking layer may have improved hole blocking ability without a substantial increase in driving voltage.

The electron transport layer may further include at least one of BOP, Bphen, Alq₃, BAlq, TAZ, and NTAZ:

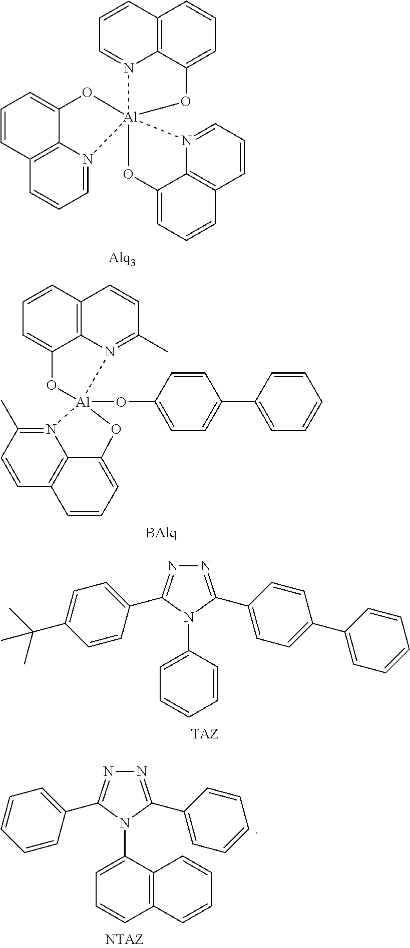

In one or more embodiments, the electron transport layer may include at least one of ET1 to ET25, but are not limited thereto:

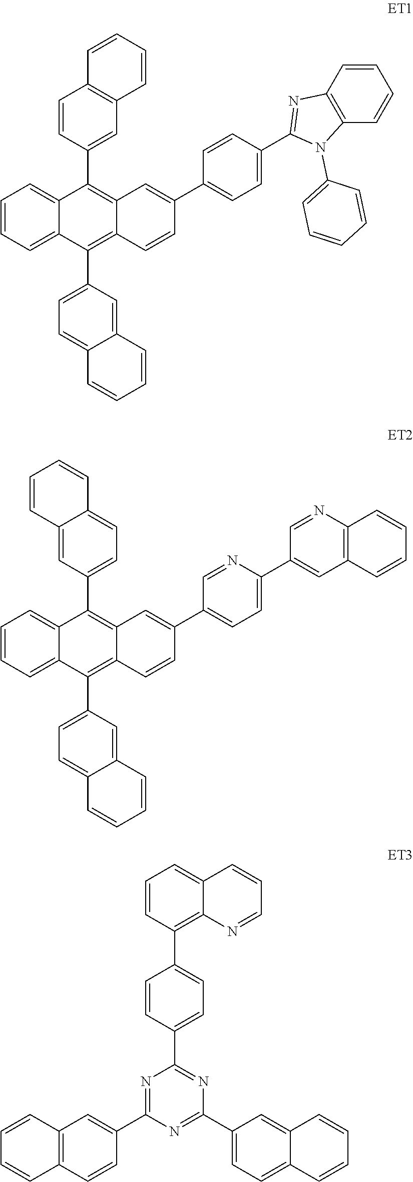 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

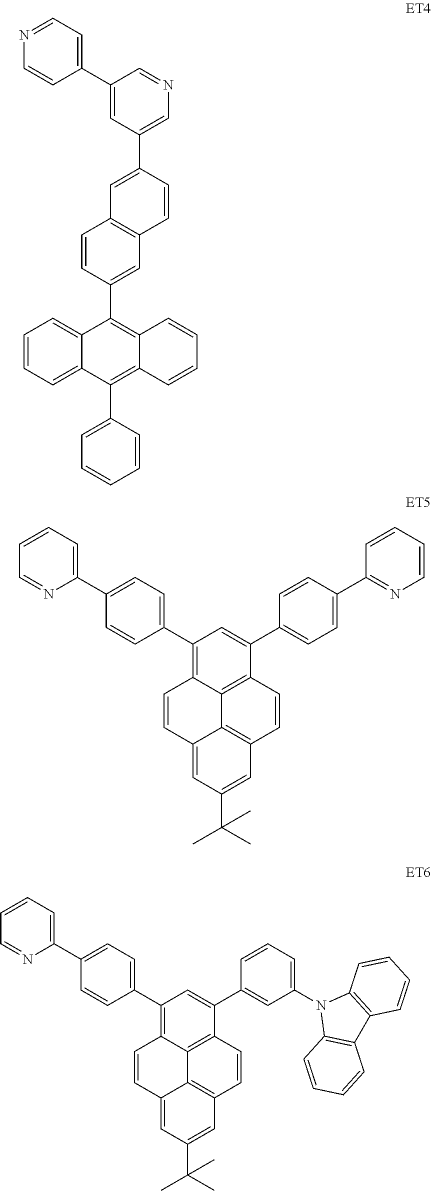 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

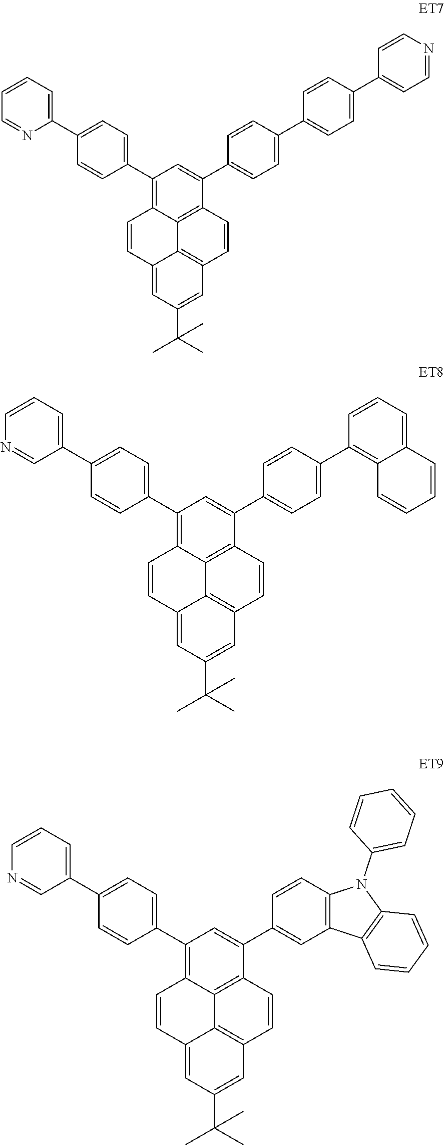 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34]

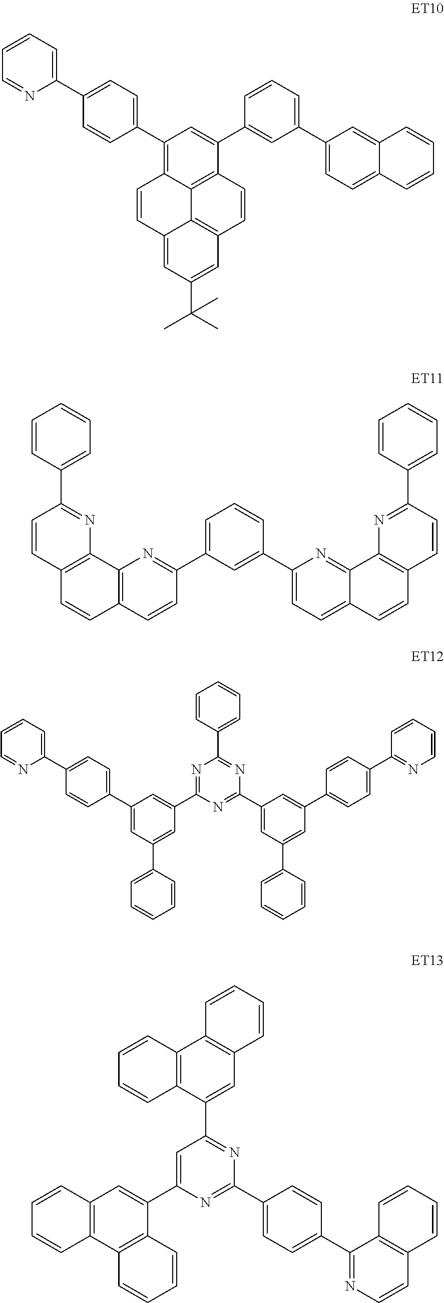 [SMILES: CC(C)(C)c1cc2ccc3c(-c4ccc(-c5ccccn5)cc4)cc(-c4cccc(-c5ccc6ccccc6c5)c4)c4ccc(c1)c2c34 ; c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc2c(-c3ccc(-c4nc(-c5cc6ccccc6c6ccccc56)cc(-c5cc6ccccc6c6ccccc56)n4)cc3)nccc2c1]

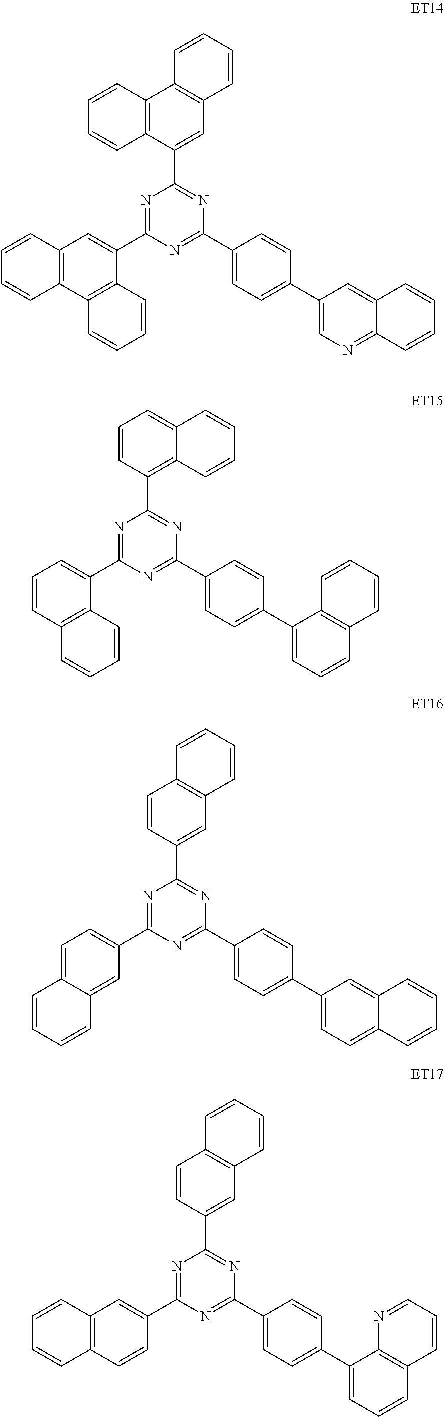 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5ccc6ccccc6c5)nc(-c5ccc6ccccc6c5)n4)cc3)ccc2c1 ; c1ccc2cc(-c3nc(-c4ccc(-c5cccc6cccnc56)cc4)nc(-c4ccc5ccccc5c4)n3)ccc2c1]

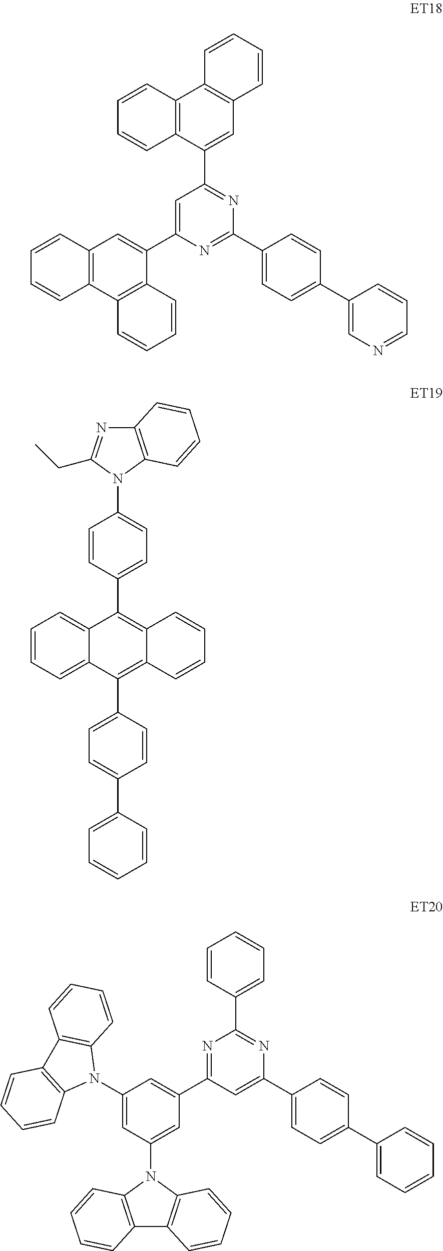 [SMILES: c1cncc(-c2ccc(-c3nc(-c4cc5ccccc5c5ccccc45)cc(-c4cc5ccccc5c5ccccc45)n3)cc2)c1 ; CCc1nc2ccccc2n1-c1ccc(-c2c3ccccc3c(-c3ccc(-c4ccccc4)cc3)c3ccccc23)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

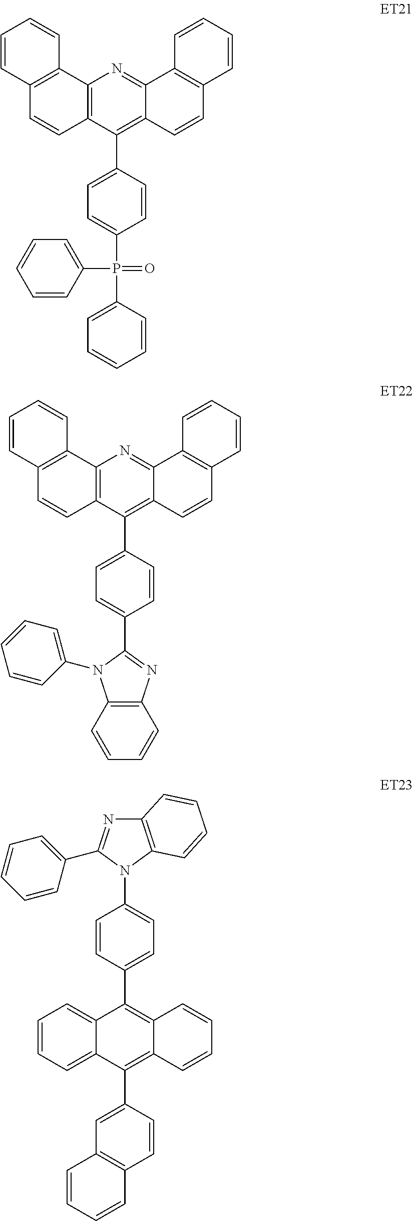

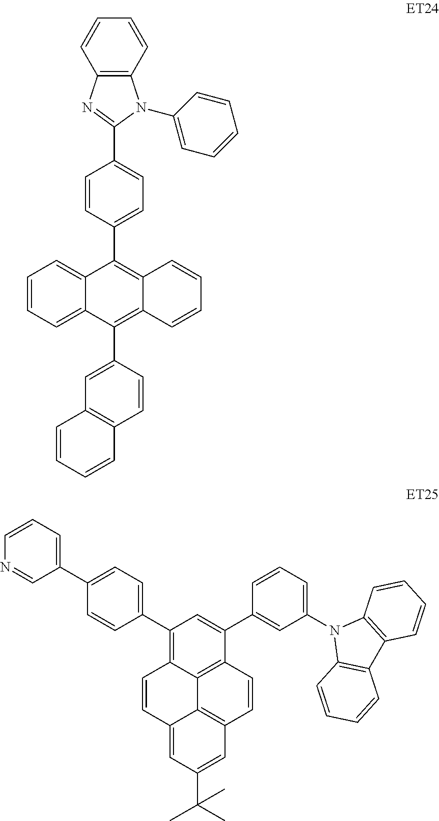 [SMILES: c1ccc(-n2c(-c3ccc(-c4c5ccccc5c(-c5ccc6ccccc6c5)c5ccccc45)cc3)nc3ccccc32)cc1 ; CC(C)(C)c1cc2ccc3c(-c4ccc(-c5cccnc5)cc4)cc(-c4cccc(-n5c6ccccc6c6ccccc65)c4)c4ccc(c1)c2c34]

A thickness of the electron transport layer may be in a range of about 100 Å to about 1,000 Å, for example, about 150 Å to about 500 Å. When the thickness of the electron transport layer is within the range described above, the electron transport layer may have satisfactory electron transport characteristics without a substantial increase in driving voltage.

Also, the electron transport layer may further include, in addition to the materials described above, a metal-containing material.

The metal-containing material may include a Li complex. The Li complex may include, for example, Compound ET-D1 (lithium 8-hydroxyquinolate, LiQ) or ET-D2:

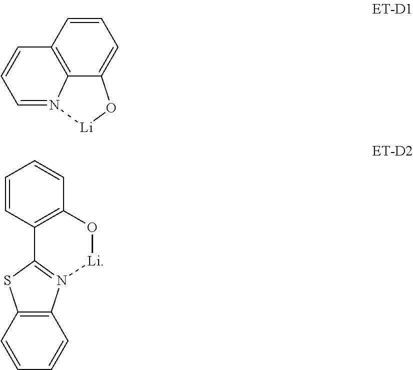

The electron transport region may include an electron injection layer that promotes flow of electrons from the second electrode 19 thereinto.

The electron injection layer may include at least one of LiF, NaCl, CsF, Li₂O, or BaO.

A thickness of the electron injection layer may be in a range of about 1 Å to about 100 Å, for example, about 3 Å to about 90 Å. When the thickness of the electron injection layer is within the range described above, the electron injection layer may have satisfactory electron injection characteristics without a substantial increase in driving voltage.

The second electrode 19 is disposed on the organic layer 15. The second electrode 19 may be a cathode. The material for forming the second electrode 19 may be a metal, an alloy, an electrically conductive compound, and a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as a material for forming the second electrode 19. In one or more embodiments, to manufacture a top-emission type light-emitting device, a transmissive electrode formed using ITO or IZO may be used as the second electrode 19.

Hereinbefore, the organic light-emitting device has been described with reference to FIG. 3, but embodiments of the present disclosure are not limited thereto.

According to another aspect, there is provided fluorescent compound,
wherein the fluorescent compound may have ³n-π*-to-¹π-π* energy transition from a ³n-π* excited state to a ¹π-π* excited state,
an energy level in a ¹n-π* excited state of the fluorescent compound is greater than an energy level in the ¹π-π* excited state of the fluorescent compound,
the fluorescent compound emits a fluorescent light by radiative energy transition of an exciton in the ¹π-π* excited state to a ground state,
“³” in the expression “³n-π*” indicates a triplet state, and “¹” in the expressions “¹n-π*” and “¹π-π*” indicates a singlet state, and
the energy level in the ¹n-π* excited state, the energy level in the ¹π-π* excited state, and the energy level in the ³n-π* excited state may be each independently calculated by using a time dependent-Density Functional Theory method (for example, a time dependent-Density Functional Theory method of the Gaussian 09 program) that is structurally optimized at a level of CAM-B3LYP/6-311+G(d,p), and
the fluorescent compound is not a coumarin-based compound represented by Formula 1′:

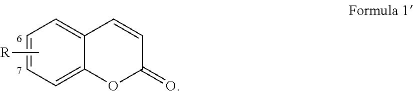

In Formula 1′, R may be a C₆-C₅₀ aryl group positioned at the 6-position or 7-position of a coumarin ring in Formula 1′. The 6-position and 7-position are identified in Formula 1′ as “6” and “7”, respectively.

The fluorescent compound may be the same as described in connection with the fluorescent compound included in the organic light-emitting device above, except that the coumarin-based compound represented by Formula 1′ is excluded from the fluorescent compound. In other words, the fluorescent compound is not the coumarin-based compound of Formula 1′.

The term “C₁-C₆₀ alkyl group” as used herein refers to a linear or branched saturated aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an isoamyl group, and a hexyl group. The term “C₁-C₆₀ alkylene group” as used herein refers to a divalent group having the same structure as the C₁-C₆₀ alkyl group.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, and an isopropyloxy group.

The term “C₂-C₆₀ alkenyl group” as used herein refers to a hydrocarbon group formed by substituting at least one double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon monocyclic group having 3 to 10 carbon atoms, and non-limiting examples thereof include a cyclopropyl group, a cyclobutyl group, a cyclopentyl group, a cyclohexyl group, and a cycloheptyl group. The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent saturated monocyclic group having at least one heteroatom that is N, O, P, Si, or S as a ring-forming atom and 1 to 10 carbon atoms, and non-limiting examples thereof include a tetrahydrofuranyl group, and a tetrahydrothiophenyl group. The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent monocyclic group that has 3 to 10 carbon atoms and at least one double bond in the ring thereof and no aromaticity, and non-limiting examples thereof include a cyclopentenyl group, a cyclohexenyl group, and a cycloheptenyl group. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom that is N, O, P, Si, or S as a ring-forming atom, 1 to 10 carbon atoms, and at least one double bond in its ring. Examples of the C₁-C₁₀ heterocycloalkenyl group are a 2,3-dihydrofuranyl group, and a 2,3-dihydrothiophenyl group. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the rings may be fused to each other.

The term “C₁-C₆₀ heteroaryl group” as used herein refers to a monovalent group having a heterocyclic aromatic system that has at least one heteroatom that is N, O, P, Si, or S as a ring-forming atom, and 1 to 60 carbon atoms. The term “C₁-C₆₀ heteroarylene group” as used herein refers to a divalent group having a heterocyclic aromatic system that has at least one heteroatom that is N, O, P, Si, or S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each include two or more rings, the rings may be fused to each other.

The term “C₆-C₆₀ aryloxy group” used herein indicates —OA₁₀₂ (wherein A₁₀₂ is the C₆-C₆₀ aryl group), and a C₆-C₆₀ arylthio group used herein indicates —SA₁₀₃ (wherein A₁₀₃ is the C₆-C₆₀ aryl group).

The term “monovalent non-aromatic condensed polycyclic group” as used herein refers to a monovalent group (for example, having 8 to 60 carbon atoms) having two or more rings condensed to each other, only carbon atoms as ring-forming atoms, and no aromaticity in its entire molecular structure (i.e., the molecular structure as a whole is non-aromatic). A non-limiting example of the monovalent non-aromatic condensed polycyclic group is a fluorenyl group. The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group (for example, having 2 to 60 carbon atoms) having two or more rings condensed to each other, at least one heteroatom that is N, O, P, Si, o S, other than carbon atoms, as a ring-forming atom, and no aromaticity in its entire molecular structure (i.e., the molecular structure as a whole is non-aromatic). A non-limiting example of the monovalent non-aromatic condensed heteropolycyclic group is a carbazolyl group. The term “divalent non-aromatic condensed heteropolycyclic group” as used herein refers to a divalent group having the same structure as the monovalent non-aromatic condensed heteropolycyclic group.

The term “C₅-C₆₀ carbocyclic group” as used herein refers to a saturated or unsaturated cyclic group having, as a ring-forming atom, 5 to 60 carbon atoms only. The C₅-C₆₀ carbocyclic group may be a monocyclic group or a polycyclic group.

The term “C₁-C₆₀ heterocyclic group” as used herein refers to a saturated or unsaturated cyclic group having, as a ring-forming atom, at least one heteroatom that is N, O, Si, P, or S other than 1 to 60 carbon atoms. The C₁-C₆₀ heterocyclic group may be a monocyclic group or a polycyclic group.

The term “carbonyl group” as used herein refers to a divalent group of the formula *—(C═O)—*′, wherein * and *′ each indicate a binding site with a neighboring atom, respectively.

At least one substituent of the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one of deuterium, —F, —Cl, —Br, —I, —CD₃, —CD₂H, —CDH₂, —CF₃, —CF₂H, —CFH₂,l a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), or —P(═O)(Q₁₈)(Q₁₉),
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), or —P(═O)(Q₂₈)(Q₂₉);

—N(Q₃₁)(Q₃₂), —Si(Q₃₃)(Q₃₄)(Q₃₅), —B(Q₃₆)(Q₃₇), or —P(═O)(Q₃₈)(Q₃₉), and

Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkyl group substituted with at least one of deuterium, a C₁-C₆₀ alkyl group, and a C₆-C₆₀ aryl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one of deuterium, a C₁-C₆₀ alkyl group, or a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group.

The term “room temperature” as used herein refers to about 25° C.

Hereinafter, a compound and an organic light-emitting device according to embodiments are described in detail with reference to Synthesis Example and Examples.

However, the organic light-emitting device is not limited thereto. The wording “B was used instead of A” used in describing Synthesis Examples means that an amount of A used was identical to an amount of B used, in terms of a molar equivalent.

### EXAMPLES

### Synthesis Example 1

### (Compound 4)

### Synthesis of Intermediate 4-1 (7-Bromocoumarin)

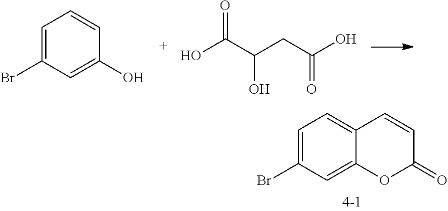

At a temperature of 0° C., 98% H₂SO₄ (6.5 mL) was added dropwise to a mixture of 3-bromophenol (5.00 g, 28.9 mmol) and DL-malic acid (2.60 g, 19.4 mmol), and the mixed solution was heated at a temperature of 120° C. for 6 hours. The crushed ice was poured into the reaction mixture thus obtained, and the precipitated solid was filtered. A filter cake collected therefrom was washed with water to remove residual H₂SO₄. Then, the resulting product was dissolved in CH₂Cl₂, dried with anhydrous MgSO₄, and concentrated under vacuum. The resulting product thus obtained was purified by silica gel chromatography using CH₂Cl_{2:}n-hexane at a ratio of 1:1 (v/v)), thereby obtaining Intermediate 4-1 (white powder, yield of 34%).

¹H NMR (400 MHz, CDCl₃) δ: 6.44 ppm (d, J=9.6 Hz, 1H), 7.33-7.43 ppm (m, 2H), 7.52 ppm (d, J=2.0 Hz, 1H), 7.66 ppm (d, J=9.6 Hz, 1H).

¹³C NMR (75 MHz, CDCl₃) δ: 117.01 ppm, 117.90 ppm, 120.29 ppm, 125.93 ppm, 128.02 ppm, 128.97 ppm, 142.92 ppm, 154.40 ppm, 160.01 ppm.

### Synthesis of Compound 4

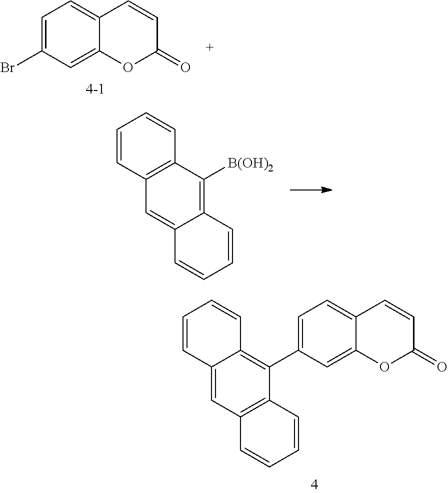

Intermediate 4-1 (0.300 g, 1.33 mmol), anthracen-10-yl-10-boronic acid (1.60 mmol), and tetrakis(triphenylphosphine)palladium (0) (0.077 g, 0.07 mmol) were added to a 100 mL round-bottom flask, and a mixture of THF and 2 normal (N) K₂CO₃ aqueous solution (2:1 (v/v)) was added thereto, and the mixed solution was refluxed for 2 days. After completion of the reaction, water was poured into the reaction mixture, and the resulting mixture was cooled. An organic layer extracted therefrom by using CH₂Cl₂ (100 mL×4 times) was dried with MgSO₄, and concentrated. The resulting product thus obtained was purified by silica gel chromatography while increasing the polarity of the eluent from CH₂Cl₂:n-hexane (at 1:2 (v/v)) to CH₂Cl₂:n-hexane (at 1:1 (v/v)), thereby obtaining Compound 4 (yellow powder, yield of 28%).

¹H NMR (300 MHz, CDCl₃) δ: 6.55 ppm (d, J=9.6 Hz, 1H), 7.35-7.41 ppm (m, 3H), 7.46-7.51 ppm (m, 3H), 7.59-7.62 ppm (m, 2H), 7.70 ppm (d, J=7.5 Hz, 1H), 7.88 ppm (d, J=9.6 Hz, 1H), 8.08 ppm (d, J=8.4 Hz, 2H), 8.56 ppm (s, 1H).

¹³C NMR (126 MHz, CDCl₃) δ: 117.00 ppm, 118.31 ppm, 119.79 ppm, 125.43 ppm, 126.12 ppm, 126.17 ppm, 127.68 ppm, 127.93 ppm, 128.69 ppm, 129.96 ppm, 131.40 ppm, 134. 62 ppm, 143.40 ppm, 143.44 ppm, 154.29 ppm, 160.87 ppm.

### Synthesis Example 2

### (Compound 1)

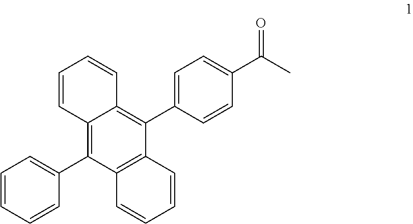 [SMILES: CC(=O)c1ccc(-c2c3ccccc3c(-c3ccccc3)c3ccccc23)cc1]

9-bromo-10-phenylanthracene (0.20 g, 0.60 mmol), 4-acetylphenylboronic acid (0.17 g, 1.02 mmol), and tetrakis(triphenylphosphine)palladium(0) (0.14 g, 0.12 mmol) were mixed with 50 mL of tetrahydrofuran (THF) at room temperature, and 25 mL of 2N K₂CO₃ (aq) was added thereto. Then, the mixed solution was heated at a temperature 70° C. for 90 hours. Water and CH₂Cl₂ were added to the reaction mixture obtained therefrom and shaken violently to extact a reaction product dissolved in a CH₂Cl₂ layer. Then, MgSO₄ was added thereto so as to remove water dissolved in the CH₂Cl₂ layer, and filtering was performed thereon by using a glass filter. The resulting product obtained therefrom was purified by silica gel column chromatography using EtOAc:n-hexane (1:3 (v/v)) to obtain Compound 1 (yellow powder, yield of 45%).

¹H NMR (300 MHz, CDCl₃) δ (ppm): 2.76 (s, 3H), 7.32-7.37 (m, 4H), 7.46-7.50 (m, 2H), 7.60-7.63 (m, 7H), 7.70-7.73 (m, 2H), 8.22 (d, J=8.7 Hz, 2H).

¹³C NMR (126 MHz, CD₂Cl₂) δ (ppm): 27.18, 125.65, 125.91, 126.95, 127.56, 128.15, 128.94, 129.00, 130.07, 130.37, 131.76, 132.23, 136.31, 137.05, 138.29, 139.37, 144.85, 198.22.

### Synthesis Example 3

### (Compound 2)

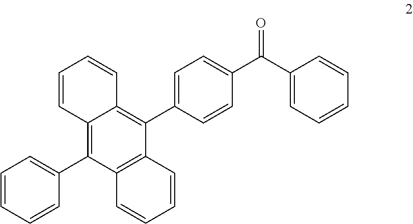 [SMILES: O=C(c1ccccc1)c1ccc(-c2c3ccccc3c(-c3ccccc3)c3ccccc23)cc1]

9-bromo-10-phenylanthracene (0.20 g, 0.60 mmol), 4-benzoylphenylboronic acid (0.28 g, 1.26 mmol), and tetrakis(triphenylphosphine)palladium(0) (0.21 g, 0.18 mmol) were mixed with 50 mL of tetrahydrofuran (THF) at room temperature, and 25 mL of 2N K₂CO₃ (aq) was added thereto. The mixed solution was heated at a temperature of 70° C. for 72 hours. Water and CH₂Cl₂ were added to the reaction mixture obtained therefrom and shaken violently to extact a reaction product dissolved in a CH₂Cl₂ layer. Then, MgSO₄ was added thereto so as to remove water dissolved in the CH₂Cl₂ layer, and filtering was performed thereon by using a glass filter. The resulting product obtained therefrom was purified by silica gel column chromatography using CH₂Cl₂:n-hexane (1:2 (v/v)) to obtain Compound 2 (yellow powder, yield of 96%).

¹H NMR (300 MHz, CDCl₃) δ (ppm): 7.33-7.40 (m, 4H), 7.47-7.50 (m, 2H), 7.55-7.66 (m, 8H), 7.67-7.74 (m, 4H), 7.97-8.00 (m, 2H), 8.08 (d, J=1.8 Hz, 2H).

¹³C NMR (126 MHz, CD₂Cl₂) δ (ppm): 30.36, 125.68, 125.92, 127.07, 127.57, 128.15, 128.94, 129.02, 130.14, 130.40, 130.61, 130.72, 131.78, 131.95, 133.05, 136.38, 137.41, 138.28, 139.39, 144.24, 196.75.

### Synthesis Example 4

### (Compound 7)

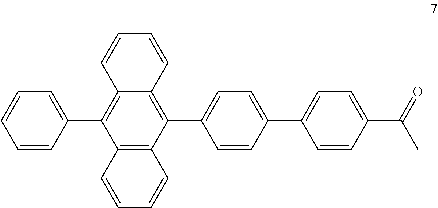 [SMILES: CC(=O)c1ccc(-c2ccc(-c3c4ccccc4c(-c4ccccc4)c4ccccc34)cc2)cc1]

9-(4-bromophenyI)-10-phenylanthracene (0.20 g, 0.49 mmol), 4-acetylphenylboronic acid (0.17 g, 1.02 mmol), and tetrakis(triphenylphosphine)palladium(0) (0.34 g, 0.29 mmol) were mixed with 50 mL of tetrahydrofuran (THF) at room temperature, and 25 mL of 2N K₂CO₃ (aq) was added thereto. The mixed solution was heated at a temperature of 70° C. for 38 hours. Water and CH₂Cl₂ were added to the reaction mixture obtained therefrom and shaken violently to extact a reaction product dissolved in a CH₂Cl₂ layer. Then, MgSO₄ was added thereto so as to remove water dissolved in the CH₂Cl₂ layer, and filtering was performed thereon by using a glass filter. The resulting product obtained therefrom was purified by silica gel column chromatography using EtOAc:n-hexane (1:2 (v/v)) to obtain Compound 7 (yellow powder, yield of 19%).

¹H NMR (300 MHz, CDCl₃) δ (ppm): 2.70 (s, 3H), 7.33-7.40 (m, 4H), 7.48-7.51 (m, 2H), 7.56-7.64 (m, 5H), 7.69-7.78 (m, 4H), 7.87-7.90 (m, 4H), 8.13 (d, J=8.4 Hz, 2H).

¹³C NMR (126 MHz, CD₂Cl₂) δ (ppm): 27.11, 125.62, 125.72, 127.29, 127.52, 127.74, 127.83, 128.11, 129.00, 129.52, 130.39, 130.44, 131.81, 132.54, 136.70, 136.98, 137.94, 139.50, 139.55, 139.68, 145.78, 197.98.

### Synthesis Example 5

### (Compound 8)

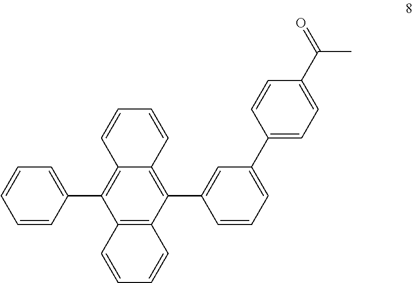 [SMILES: CC(=O)c1ccc(-c2cccc(-c3c4ccccc4c(-c4ccccc4)c4ccccc34)c2)cc1]

9-(3-bromophenyl)-10-phenylanthracene (0.20 g, 0.49 mmol), 4-acetylphenylboronic acid (0.16 g, 0.98 mmol), and tetrakis(triphenylphosphine)palladium(0) (0.23 g, 0.20 mmol) were dissolved in 50 mL of tetrahydrofuran (THF) at room temperature, and 25 mL of 2N K₂CO₃ (aq) was added thereto. The mixed solution was heated at a temperature of 70° C. for 96 hours. The mixed solution was heated at a temperature of 70° C. for 72 hours. Water and CH₂Cl₂ were added to the reaction mixture obtained therefrom and shaken violently to extact a reaction product dissolved in a CH₂Cl₂ layer. Then, MgSO₄ was added thereto so as to remove water dissolved in the CH₂Cl₂ layer, and filtering was performed thereon by using a glass filter. The resulting product obtained therefrom was purified by silica gel column chromatography using EtOAc:n-hexane (1:7 (v/v)) to obtain Compound 8 (yellow powder, yield of 86%).

¹H NMR (300 MHz, CD₂Cl₂) δ (ppm): 2.61 (s, 3H), 7.33-7.39 (m, 4H), 7.47-7.50 (m, 2H), 7.52-7.55 (m, 1H), 7.58-7.64 (m, 3H), 7.68-7.72 (m, 3H), 7.73-7.79 (m, 2H), 7.77-7.83 (m, 3H), 7.87-7.90 (m, 1H), 8.01-8.05 (m, 2H).

¹³C NMR (126 MHz, CD₂Cl₂) δ (ppm): 27.05, 30.25, 125.63, 125.76, 126.91, 127.31, 127.50, 127.79, 128.11, 129.00, 129.41, 129.70, 130.43, 130.65, 131.78, 131.80, 136.67, 137.14, 137.96, 139.48, 140.36, 140.56, 145.73, 197.91.

### Evaluation Example 1

First, regarding Compounds 1 to 4, 7, and 8, an energy level in a singlet excited state and an energy level in a triplet excited state for each compound was calculated by using the Gaussian 09 program based on Density Functional Theory (DFT)-dependent quantum chemistry calculation. The results determining an energy level in a ¹n-π* excited state, an energy level in a ¹π-π* excited state, an energy level in a ³n-π* excited state, and an energy level in a ³π-π* excited state are shown in Table 1.

Here, geometry optimization and single point calculation of model structures were performed using the long range corrected version of B3LYP using the Coulomb-attenuating method (CAM-B3LYP) and 6-311+G(d,p) basis set. A polarizable continuum model (CPCM) parameterized for THF was applied during the geometry optimization step. Then, frequency calculation was performed in order to assess stability of convergence. Time-dependent-Density Functional Theory (TD-DFT) calculation was applied to the optimized geometries using the same functionals and basis sets that were used for geometry optimization. The CPCM parameterized for THF was applied to account for salvation effects. The twenty lowest singlet and triplet states were calculated and analyzed.

Meanwhile, the non-bonding and the 1T orbitals were determined based on the topologies thereof, and the oscillator strengths and the expansion coefficients were obtained by a logarithmic file.

TABLE 1

| Compound No. | 1 | 2 | 3 | 4 | 7 | 8 |
|---|---|---|---|---|---|---|
| Energy level (eV) in ¹n-π* excited state | 3.78 | 3.82 | 2.05 | 4.06 | 3.78 | 3.78 |
| Energy level (eV) in ¹π-π* excited state | 3.52 | 3.58 | 1.35 | 3.80, 3.11 | 3.51 | 3.52 |
| Energy level (eV) in ³n-π* excited state | 3.43 | 3.20 | 2.04 | 2.81 | 3.43 | 3.44 |
| Energy level (eV) in ³π-π* excited state | 3.21 | 2.21 | 1.68, 0.78, | 3.46, 3.29 | 2.21 | 2.21 |

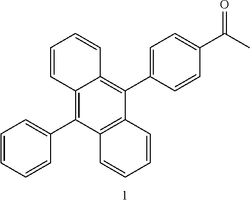 [SMILES: CC(=O)c1ccc(-c2c3ccccc3c(-c3ccccc3)c3ccccc23)cc1]

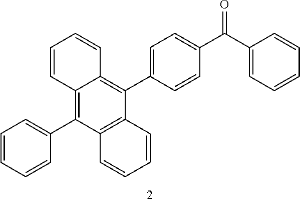 [SMILES: O=C(c1ccccc1)c1ccc(-c2c3ccccc3c(-c3ccccc3)c3ccccc23)cc1]

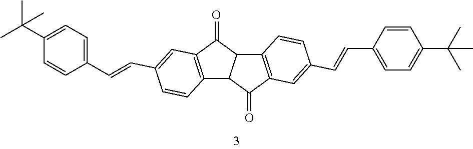 [SMILES: CC(C)(C)c1ccc(/C=C/c2ccc3c(c2)C(=O)C2c4ccc(/C=C/c5ccc(C(C)(C)C)cc5)cc4C(=O)C32)cc1]

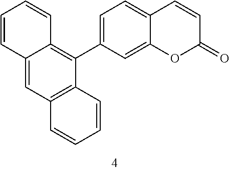 [SMILES: O=c1ccc2ccc(-c3c4ccccc4cc4ccccc34)cc2o1]

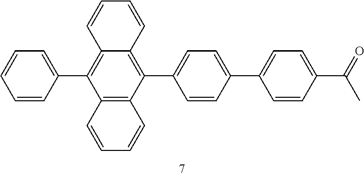 [SMILES: CC(=O)c1ccc(-c2ccc(-c3c4ccccc4c(-c4ccccc4)c4ccccc34)cc2)cc1]

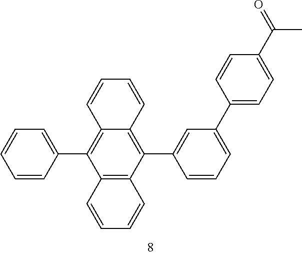 [SMILES: CC(=O)c1ccc(-c2cccc(-c3c4ccccc4c(-c4ccccc4)c4ccccc34)c2)cc1]

Referring to Table 1, it was confirmed that, in Compounds 1 to 4, 7, and 8, the energy level in the ¹n-π* excited state was higher than the energy level in the ¹π-π* excited state.

### Evaluation Example 2

A quartz substrate washed with acetone, isopropyl alcohol, and pure water was prepared, and then, predetermined materials shown in Table 2 were vacuum-(co)-deposited thereon at a vacuum of 10⁻⁷ torr, thereby preparing Films 1, A, B, and C, each having a thickness of 50 nanometers (nm).

TABLE 2

| Film No. | Compounds used for film preparation |
|---|---|
| Film 1 | Compound 4 and mCBP (at a volume ratio of 15:85) |
| Film A | BDpyInCz and mCBP (at a volume ratio of 15:85) |
| Film B | Ir—C and mCBP (at a volume ratio of 15:85) |
| Film C | ACR and mCBP (at a volume ratio of 15:85) |

The structures of Compounds 4, BDpyInCz, Ir—C, and ACR are provided below:

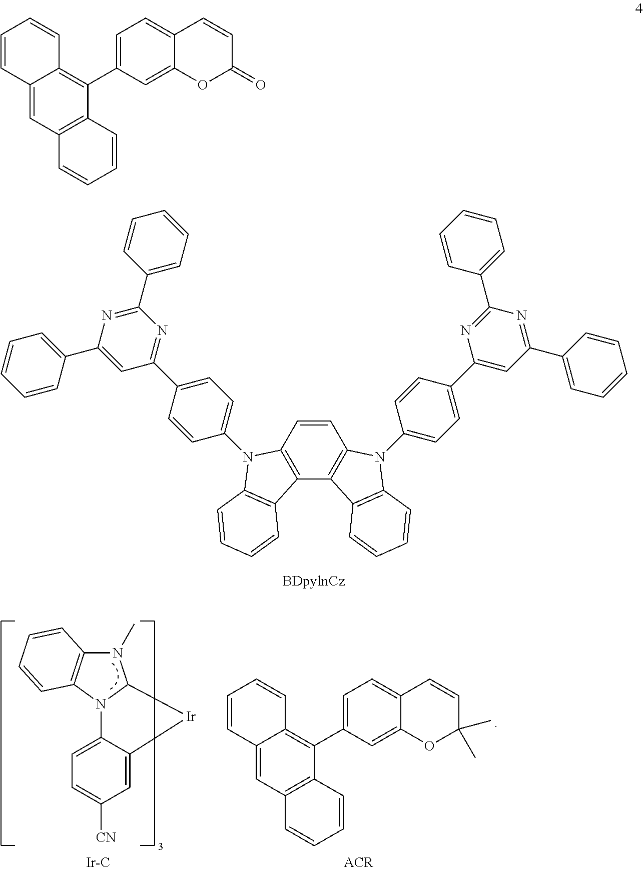

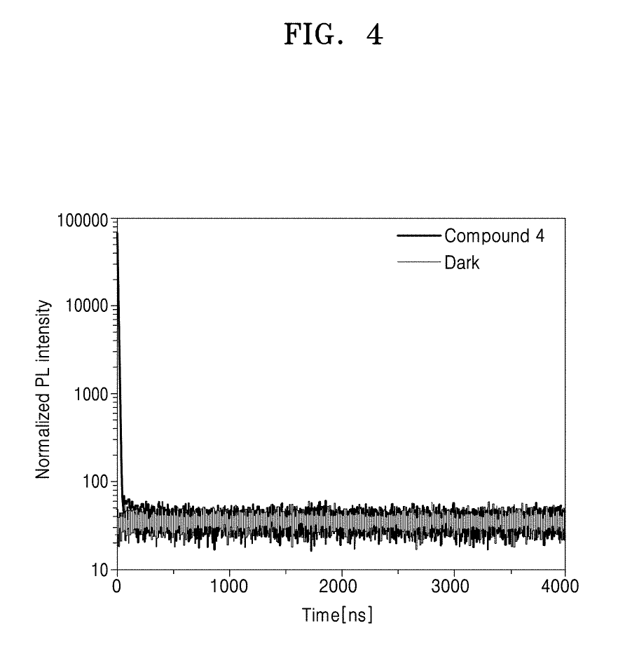

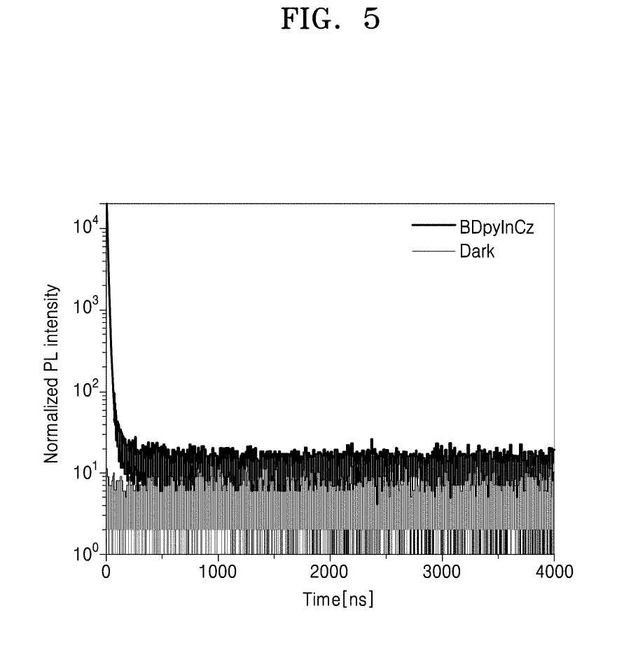

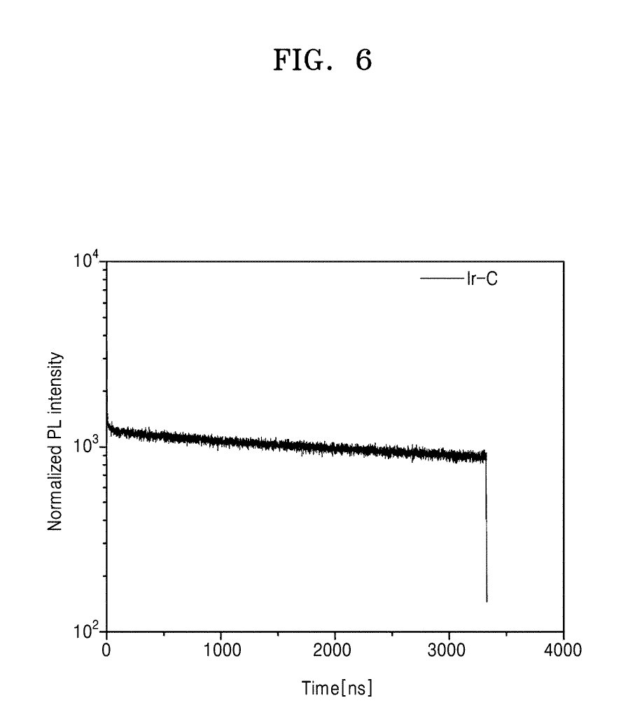

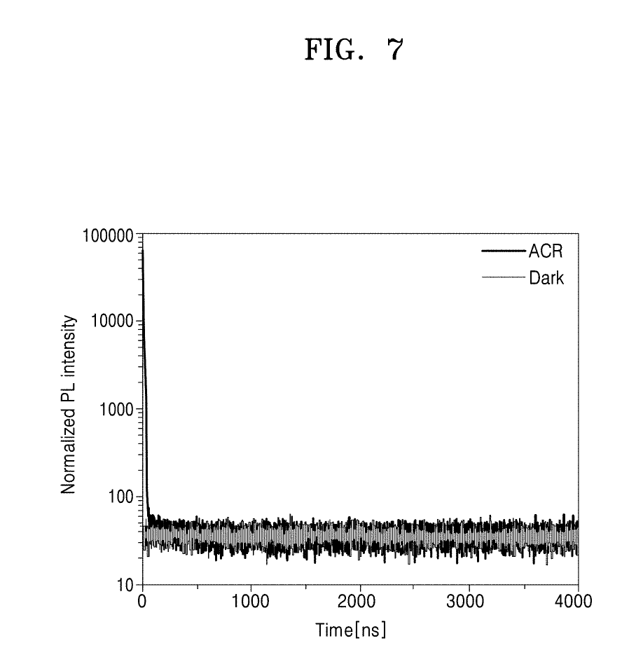

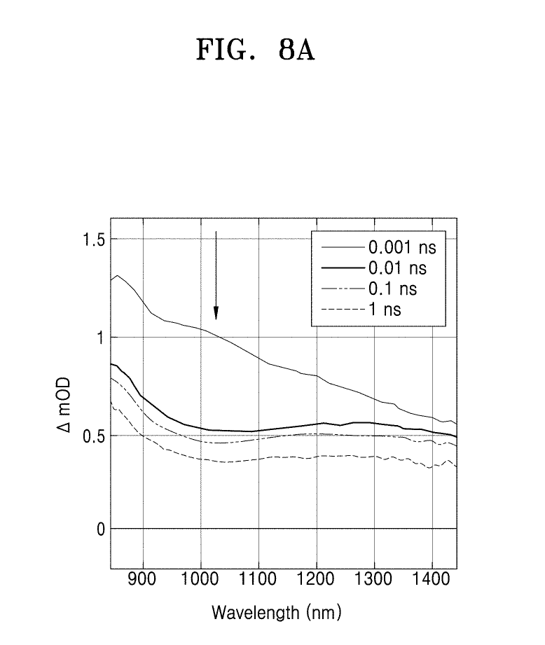

For each of Films 1, A, B, and C, the photoluminescence (PL) spectrum was evaluated at room temperature using FluoTime 300, which is a TRPL measurement system manufactured by PicoQuant Company, and manufactured by PLS340 (exciton wavelength=340 nanometers, spectral width=20 nanometers), which is a pumping source available from PicoQuant. Then, the wavelength of the main peak of the PL spectrum thus obtained was determined, and the number of photons emitted at respective wavelengths of the main peak upon the photon pulse (pulse width=500 picoseconds) applied to each film by the PLS340 was repeatedly measured in a time-dependent manner based on the time-correlated single photon counting (TCSPC). Accordingly, TRPL curves capable of having enough fitting were obtained and are shown in FIG. 4 (Film 1 (Compound 4)), FIG. 5 (Film A (BDpyInCz)), FIG. 6 (Film B Or—C)), and FIG. 7 (Film C (ACR)).

It was confirmed that BDpyInCz was able to emit delayed fluorescence based on the TRPL curve in FIG. 5 (Film A (BDpyInCz), Ir—C was able to emit phosphorescence based on the TRPL curve in FIG. 6 (Film B(Ir—C)), and Compound 4 and ACR were each able to emit general fluorescence based on the TRPL curves in FIG. 4 (Film 1 (Compound 4)) and FIG. 7 (Film C (ACR)).

Subsequently, the decay times of Films 1, A, B, and C were determined by fitting two or more exponential decay functions to the results obtained from the TRPL curves, and the results are shown in Table 3. An exponential function used for the fitting is as shown in Equation 1, and the largest value among values obtained from each exponential decay functional used in the fitting was taken as the decay time for each film. Here, during the same measurement time as the measurement time taken to obtain the TRPL curves, the same measurement was repeated one more time in dark (i.e., a state in which a pumping signal incident on the predetermined film is blocked), so as to obtain a baseline or background signal curve to be used as a baseline in the fitting. In the case of Film A, among a curve of a prompt fluorescent emission substance and a curve of a delayed fluorescent emission substance, a curve of a delayed fluorescent emission substance was used to calculate decay time thereof:

$$f ( t ) = {\sum }_{i = 1}^{n} {A}_{i} exp ( - t / {T}_{decay , i} ) \qquad Equation 1$$

TABLE 3

| Film No. | Decay time (exciton lifetime) | |
|---|---|---|
| Film 1 (Compound 4) | 6 | ns |
| Film A (BDpyInCz) | 32 | μs |
| Film B (Ir—C) | 14 | μs |
| Film C (ACR) | 6 | ns |

Referring to Table 3, it was confirmed that the exciton lifetime of Compound 4 was significantly shorter than that of BDpyInCz (TADF) and Ir—C (phosphorescence), but was similar with that of ACR (fluorescence).

### Evaluation Example 3

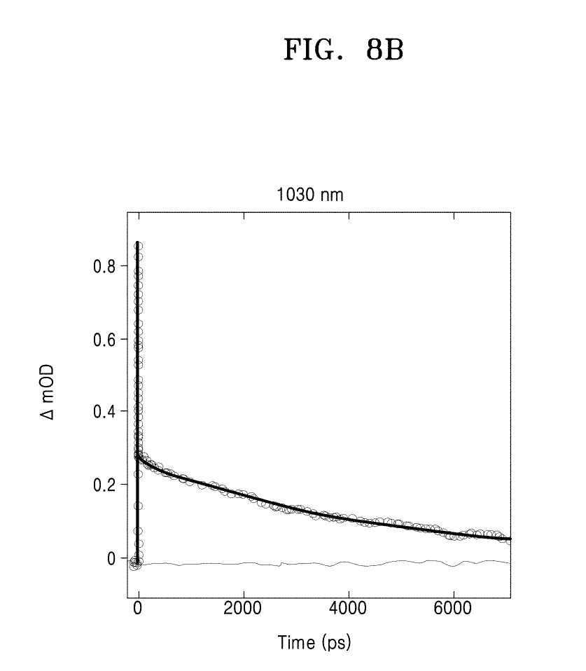

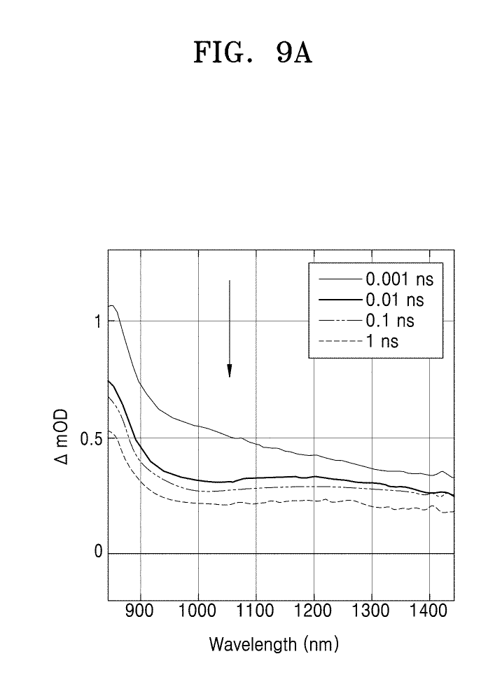

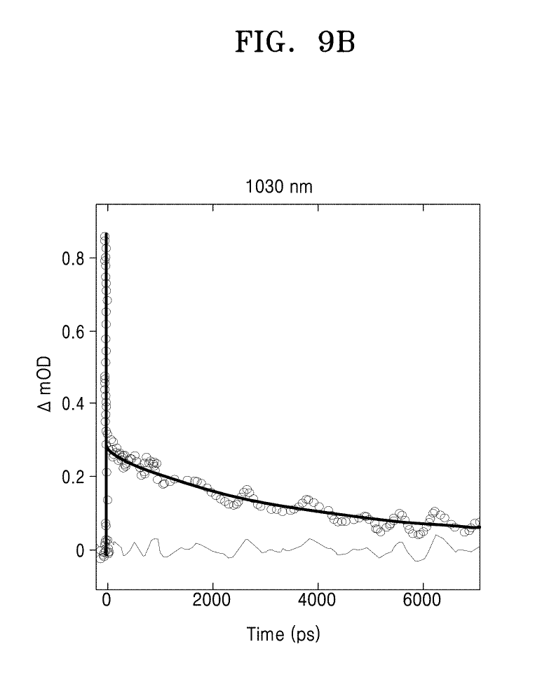

Transient absorption spectra of Compound 4 were measured at room temperature by using a home-built pump-probe system, and results thereof and analysis data are shown in FIGS. 8A, 8B, 9A, and 9B, and in Table 4.

As a test sample, an acetonitrile solution (200 micromolar concentration) of Compound 4 was prepared, transient absorption spectrum measurement for N₂-saturated acentonitrile solution of Compound 4 was performed after bubbling of 30 minutes under N₂ purging, and transient absorption spectrum measurement for air-saturated acetonitrile solution of Compoun 4 was performed without N₂ purging.

A 320 nm pumping beam of a transient absorption spectrum measurement device was obtained from an optical parametric amplifier (Light conversion, TOPAS-C) pumped by a regerneratively amplified Ti:Sapphire laser system (Coherent, Libra, 800 nm wavelength, 50 fs pulse duration, 1 kHz repetition rate). A probe beam is a white light continuum generated by focusing a small portion of the amplifier output though a sapphire window.

FIG. 8A is a transient absorption spectrum obtained when an absorption variation (ΔmOD, a.u.) of an N₂-saturated acetonitrile solution of Compound 4 with respect to a wavelength (nm) is measured at 0.001 ns, 0.01 ns, 0.1 ns, and 1 ns after photoexcitation, respectively, and FIG. 9A is a transient absorption spectrum obtained when an absorption variation (ΔmOD, a.u.) of an air-saturated acetonitrile solution of Compound 4 with respect to a wavelength (nm) is measured at 0.001 ns, 0.01 ns, 0.1 ns, and 1 ns after photoexcitation, respectively.

Considering that the absorption variation at the wavelength of 1030 nm increases in the graph measured at 0.001 ns after photoexcitation in FIG. 8A (see an arrow in FIG. 8A) 1) the absorption variation (ΔmOD, a.u.) of the N₂-saturated acetonitrile solution of Compound 4 and the air-saturated acetonitrile solution of Compound 4 with respect to the time (ps) for the wavelength of 1030 nm was measured, and results thereof are shown in FIGS. 8B and 9B, and 2) two absorption decay times that could be observed from FIGS. 8B and 9B, that is, τ₂ and τ₃, were evaluated, and results thereof are shown in Table 4.

TABLE 4

| | τ₂ (ps) | τ₃ (ns) |
|---|---|---|
| N₂-saturated acetonitrile | 41 | 4.1 |
| solution of Compound 4 | | |
| air-saturated acetonitrile | 23 | 2.9 |
| solution of Compound 4 | | |

Referring to Table 4, τ₂ of the air-saturated acentonitrile solution of Compound 4 with respect to the light of 1,030 nm was more reduced than τ₂ of the N₂-saturated acetonitrile solution of Compound 4 with respect to the light of 1,030 nm, and τ₃ of the air-saturated acentonitrile solution of Compound 4 with respect to the light of 1,030 nm was more reduced than τ₃ of the N₂-saturated acetonitrile solution of Compound 4 with respect to the light of 1,030 nm. By this Table 4, a remarkable difference in absorption decay time according to the presence or absence of oxygen was confirmed. Therefore, the excited state of Compound 4 observed at the wavelength of 1,030 nm in FIG. 8A may be determined as not a singlet state but a triplet state.

On the other hand, since 3.8 eV, which is the pump energy of the pump beam of 320 nm used for optical excitation, is equal to the energy level of the ¹π-π* excited state of Compound 4 (see Table 1), the initial excited state of Compound 4 may be determined as the ¹π-π* excited state. The excited state of Compound 4 at the wavelength of 1,030 nm (determined as the triplet state as described above) may be observed from the transient absorption spectrum of FIG. 8A (that is, the absorption variation at the wavelength of 1,030 nm increases). This transient absorption spectrum of FIG. 8A may be an evidence for a transition of more than a part of energy of the ¹π-π* excited state generated by photoexcitation to the excited state at the wavelength of 1,030 nm. In summary, according to EI-Sayed rule, the excited state of Compound 4 at the wavelength of 1,030 nm may be determined as not the triplet state of the ground state but the ³n-π* excited state. Therefore, it is confirmed that Compound 4 may have the ³n-π* excited state. In Table 4, τ₂ (in tens ps order) may be defined as the absorption decay time observed when Compound 4 transits from the ¹π-π* excited state to the ³n-π* excited state, and τ3 (in numbers ns order) may be defined as the absorption decay time observed when Compound 4 transits from the ¹π-π* excited state to the lowest triplet state.

### Example 1

As an anode, a glass substrate on which an ITO electrode was formed was cut to a size of 50 mm×50 mm×0.5 mm, sonicated with acetone isopropyl alcohol and pure water each for 15 minutes, and then, cleaned by exposure to ultraviolet rays for 30 minutes.

Compounds HT3 and HT-D2 (HT-D2 having a concentration of 3 wt %) were co-deposited on the anode to form a hole injection layer having a thickness of 100 Angstroms (Å). Compound HT3 was deposited on the hole injection layer to form a hole transport layer having a thickness of 1,500 Å, and mCP was deposited on the hole transport layer to form an electron blocking layer having a thickness of 100 Å, thereby forming a hole transport region having a thickness of 1,700 Å.

Compound mCBP (host) and Compound 4 (dopant) were co-deposited on the hole transport region at a volume ratio of 85:15, thereby forming an emission layer having a thickness of 400 Å.

Compound BCP was vacuum-deposited on the emission layer to form a hole blocking layer having a thickness of 100 Å, and Compound ET17 and LiQ were co-deposited on the hole blocking layer at a weight ratio of 5:5 to form an electron transport layer having a thickness of 360 Å. Then, LiQ was deposited on the electron transport layer to form an electron injection layer having a thickness of 5 Å, and Al was formed on the electron injection layer, thereby completing the manufacture of an organic light-emitting device:

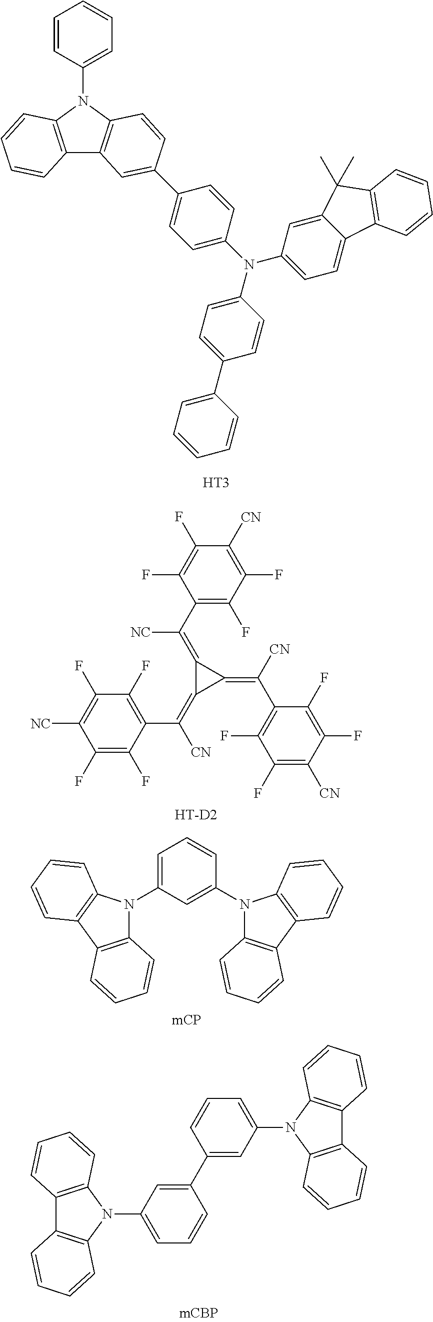

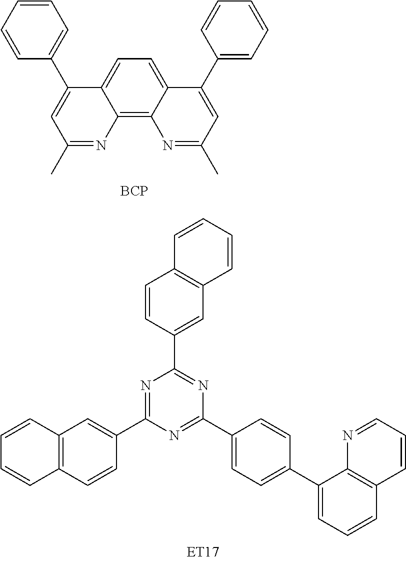

### Examples 2 to 5 and Comparative Examples A and B

An organic light-emitting device was manufactured in the same manner as in Example 1, except that a material shown in Tables 5 and 6,respectively, were used instead of the dopant in forming an emission layer.

### Evaluation Example 4

The brightness, emission efficiency (candela per ampere, Cd/A), maximum external quantum efficiency (EQEₘₐₓ, %), photoluminescence quantum yield (PLQY, %), internal quantum efficiency (IQE, %)), and lifetime (T₈₀, hours (hr)) of the organic light-emitting devices of Examples 1 to 5 and Comparative Examples A and B were measured by using Keithley 2400 current-voltage meter and Minolta Cs-1000A luminance meter, and the results are summarized in Tables 5 and 6. The lifetime (T₈₀) (at 500 Cd/m²) was a time (in hours) required to degrade brightness to 80% of an initial brightness (100%), and the IQE/PLQY was calculated under the assumption that the outcoupling efficiency of the organic light-emitting device was 0.2264.

TABLE 5

| | | | Emission | | | | | Lifetime |
|---|---|---|---|---|---|---|---|---|
| | | Brightness | efficiency | EQEₘₐₓ | PLQY | IQE | IQE/PLQY | (T₈₀) |
| | Dopant | (cd/m²) | (cd/A) | (%) | (%) | (%) | (%) | (hr) |
| Example 1 | Compound 4 | 500 | 3.31 | 3.3 | 47.9 | 16.5 | 30.4 | 1.77 |
| Comparative | ACR | 500 | 0.96 | 2.9 | 51.4 | 14.5 | 25 | 0.04 |
| Example A | | | | | | | | |

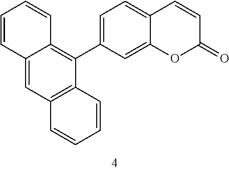 [SMILES: O=c1ccc2ccc(-c3c4ccccc4cc4ccccc34)cc2o1]

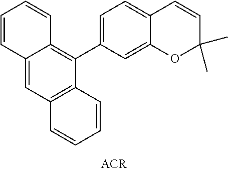 [SMILES: CC1(C)C=Cc2ccc(-c3c4ccccc4cc4ccccc34)cc2O1]

TABLE 6

| | | | Emission | | | | |
|---|---|---|---|---|---|---|---|
| | | Brightness | efficiency | EQEₘₐₓ | PLQY | IQE | IQE/PLQY |
| | Dopant | (cd/m²) | (cd/A) | (%) | (%) | (%) | (%) |
| Example 2 | Compound 1 | 500 | 1.5 | 3.7 | 48.9 | 18.7 | 38.3 |
| Example 3 | Compound 2 | 500 | 1.4 | 2.6 | 28.4 | 12.9 | 45.5 |
| Example 4 | Compound 7 | 500 | 2.3 | 4.8 | 54.2 | 24.1 | 44.4 |
| Example 5 | Compound 8 | 500 | 1.2 | 3.8 | 59.4 | 19.2 | 32.4 |
| Comparative | DPA | 500 | 1.2 | 3.9 | 72.4 | 19.3 | 26.6 |
| Example B | | | | | | | |

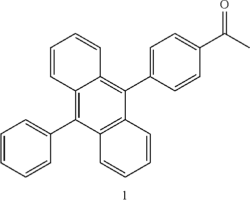 [SMILES: CC(=O)c1ccc(-c2c3ccccc3c(-c3ccccc3)c3ccccc23)cc1]

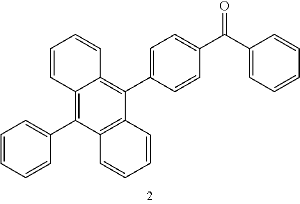 [SMILES: O=C(c1ccccc1)c1ccc(-c2c3ccccc3c(-c3ccccc3)c3ccccc23)cc1]

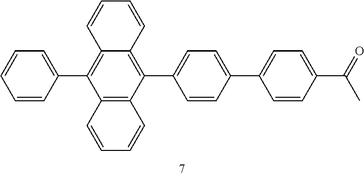 [SMILES: CC(=O)c1ccc(-c2ccc(-c3c4ccccc4c(-c4ccccc4)c4ccccc34)cc2)cc1]

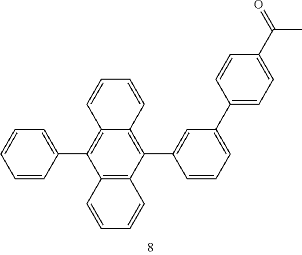 [SMILES: CC(=O)c1ccc(-c2cccc(-c3c4ccccc4c(-c4ccccc4)c4ccccc34)c2)cc1]

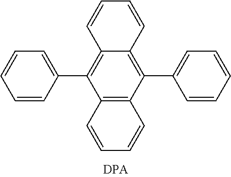 [SMILES: c1ccc(-c2c3ccccc3c(-c3ccccc3)c3ccccc23)cc1]

Referring to Tables 5 and 6, it was confirmed that the organic light-emitting device of Example 1 exhibited both excellent emission efficiency and longer lifetime as compared with the organic light-emitting device of Comparative Example A and the organic light-emitting devices of Examples 2 to 5 exhibited excellent emission efficiency as compared with the organic light-emitting device of Comparative Example B. Although not particularly limited to a specific theory, the organic light-emitting device of Examples 1 to 5 exhibited the spin statics (i.e., IQE/PLQY) which is significantly higher than the spin statics of about 25% seen in a conventional fluorescence device (i.e., the organic light-emitting device of Comparative Examples A and B), confirming that fluorescence utilizing triplet excitons in the ³n-π* excited state was available, unlike a conventional fluorescence devices.

Furthermore, referring to Evaluation Example 2, it was also confirmed that Compound 4 showed the decay time at a fluorescence emission level. Thus, it was confirmed that, unlike conventional phosphorescence emission or TADF emission, Compound 4 had a mechanism of emitting light after rapid transition, which is allowed according to the El-Sayed's Rule, from the ³n-π* excited state to the ¹π-π* excited state.

In the fluorescent compound, since the fluorescence (i.e., the fluorescent light) is emitted by radiative energy transition of the exciton thereof in the ¹π-π* excited state, which is transferred from the ³n-π* excited state of the fluorescent compound to the ¹π-π* excited state of the fluorescent compound, to a ground state, the triplet excitons can contribute to fluorescence emission. Accordingly, the fluorescent compound may be able to provide high luminance and/or high emission efficiency. At the same time, the fluorescent compound can also emit fluorescence by transferring of the triplet exciton in the ³n-π* excited state to the ¹π-π* excited state via the allowed transition according to the EI-Sayed's Rule, thereby having a relatively short exciton lifetime (i.e., decay time). In this regard, the fluorescent compound may have a low probability of deterioration, and an electronic device, for example, an organic light-emitting device including the fluorescent compound may have a long lifetime. In addition, the since the fluorescent compound was not an expensive metal-containing compound, using such as iridium, platinum, or the like, an electronic device, for example, an organic light-emitting device, including the fluorescent compound may not cause an increase in the manufacturing cost thereof.

It should be understood that embodiments described herein should be considered in a descriptive sense and not for purposes of limitation. Descriptions of features or aspects within each embodiment should be considered as available for other features or aspects in other embodiments.

While one or more embodiments have been described with reference to the figures, it will be understood by those of ordinary skill in the art that various changes in form and details may be made therein without departing from the spirit and scope as defined by the following claims.

What is claimed is:

1. An organic light-emitting device comprising:
a first electrode;
a second electrode facing the first electrode; and
an organic layer disposed between the first electrode and the second electrode,
wherein the organic layer comprises an emission layer and a fluorescent compound,
wherein the fluorescent compound comprises a ³n-π*-to-¹π-π* energy transition from a ³n-π* excited state to a ¹π-π* excited state,
an energy level in a ¹n-π* excited state of the fluorescent compound is greater than an energy level in the ¹π-π* excited state of the fluorescent compound,
the fluorescent compound emits a fluorescent light by radiative energy transition of an exciton in the ¹π-π* excited state to a ground state,
“³” in the expression “³n-π*” indicates a triplet state, and “¹” in the expressions “¹n-π*” and “¹π-π*” indicates a singlet state, and
the energy level in the ¹n-π* excited state, the energy level in the ¹π-π* excited state, and the energy level in the ³n-π* excited state are each independently calculated by using a time dependent-Density Functional Theory method that is structurally optimized at a level of CAM-B3LYP/6-311+G(d,p).

2. The organic light-emitting device of claim 1, wherein an energy level in the ³n-π* excited state of the fluorescent compound is greater than the energy level in the ¹π-π* excited state of the fluorescent compound.

3. The organic light-emitting device of claim 1, wherein an energy level in the ³n-π* excited state of the fluorescent compound is less than the energy level in the ¹π-π* excited state of the fluorescent compound.

4. The organic light-emitting device of claim 1, wherein a difference between i) an energy level of the ³n-π* excited state of the fluorescent compound and ii) a lowest energy level in the ¹π-π* excited state of the fluorescent compound is about 1 electron Volt or less.

5. The organic light-emitting device of claim 1, wherein an exciton in the ³n-π* excited state of the fluorescent compound is transferred to the ¹π-π* excited state of the fluorescent compound via reverse intersystem crossing.

6. The organic light-emitting device of claim 1, wherein the fluorescent light is emitted by radiative energy transition of the exciton in the ¹π-π* excited state to the ground state, which is transferred from the ³n-π* excited state of the fluorescent compound to the ¹π-π* excited state of the fluorescent compound via reverse intersystem crossing.

7. The organic light-emitting device of claim 6, wherein a rate of the reverse intersystem crossing is in a range of about 10⁶ inverse seconds to about 10⁸ inverse seconds.

8. The organic light-emitting device of claim 1, wherein the fluorescent compound has an exciton lifetime in a range of about 0.1 nanoseconds to about 1 microseconds.

9. The organic light-emitting device of claim 1, wherein
an energy level in the ³n-π* excited state of the fluorescent compound is less than an energy level in a ³π-π* excited state of the fluorescent compound,
“³” in the expression “³π-π*” indicates a triplet state, the energy level in the ³π-π* excited state is calculated by using the time dependent-Density Functional Theory method that is structurally optimized at the level of CAM-B3LYP/6-311+G(d,p).

10. The organic light-emitting device of claim 1, wherein the fluorescent compound comprises a non-bonding molecular orbital that induces the ³n-π*-to-¹π-π* transition from the ³n-π* excited state to the ¹π-π* excited state.

11. The organic light-emitting device of claim 1, wherein the fluorescent compound comprises at least one carbonyl group.

12. The organic light-emitting device of claim 1, wherein the fluorescent compound is represented by Formula 1 or 2: 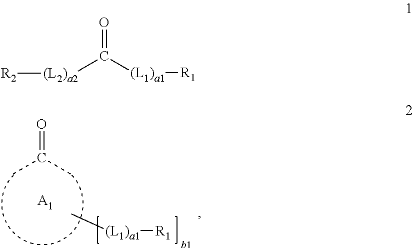
wherein in Formulae 1 and 2,
ring A₁ is a carbonyl-containing C₅-C₅₀ carbocyclic group or a carbonyl-containing C₁-C₆₀ heterocyclic group,
each L₁ and L₂ are the same or different, and are each independently a substituted or unsubstituted C₁-C₆₀ alkylene group, a substituted or unsubstituted C₂-C₆₀ alkenylene group, a substituted or unsubstituted C₂-C₆₀ alkynylene group, a substituted or unsubstituted C₃-C₁₀ cycloalkylene group, a substituted or unsubstituted C₁-C₁₀ heterocycloalkylene group, a substituted or unsubstituted C₃-C₁₀ cycloalkenylene group, a substituted or unsubstituted C₁-C₁₀ heterocycloalkenylene group, a substituted or unsubstituted C₆-C₆₀ arylene group, a substituted or unsubstituted C₁-C₆₀ heteroarylene group, a substituted or unsubstituted divalent non-aromatic condensed polycyclic group, or a substituted or unsubstituted divalent non-aromatic condensed heteropolycyclic group,
a1 and a2 are each independently an integer from 0 to 20,
R₁ and R₂ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —B(Q₆)(Q₇), —or P(═O)(Q₈)(Q₉),
b1 is an integer from 1 to 20,
at least one substituent of the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, substituted divalent non-aromatic condensed polycyclic group, substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one of deuterium, —F, —Cl, —Br, —I, —CD₃, —CD2H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —B(Q₁₆)(Q₁₇), or —P(═O)(Q₁₈)(Q₁₉), a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one of deuterium, —F, —Cl, —Br, —I, —CD₃, —CD₂H, —CDH₂, —CF₃, —CF₂H, —CFH₂, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —B(Q₂₆)(Q₂₇), or —P(═O)(Q₂₈)(Q₂₉); or
—N(Q₃₁)(Q₃₂), —Si(Q₃₃)(Q₃₄)(Q₃₅), —B(Q₃₆)(Q₃₇), or —P(═O)(Q₃₈)(Q₃₉), and
Q₁ to Q₉, Q₁₁ to Q₁₉, Q₂₁ to Q₂₉, and Q₃₁ to Q₃₉ are each independently hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₁-C₆₀ alkyl group substituted with at least one of deuterium, a C₁-C₆₀ alkyl group, and a C₆-C₆₀ aryl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryl group substituted with at least one of deuterium, a C₁-C₆₀ alkyl group, or a C₆-C₆₀ aryl group, a C₆-C₆₀ aryloxy group, a C₆-₆₀ arylthio group, a C₁-C₆₀ heteroaryl group, a monovalent non-aromatic condensed polycyclic group, or a monovalent non-aromatic condensed heteropolycyclic group.

13. The organic light-emitting device of claim 12, wherein a core represented by the structure 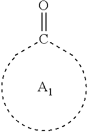
in Formula 2 is a group represented by one of Formulae 2-1 to 2-15:

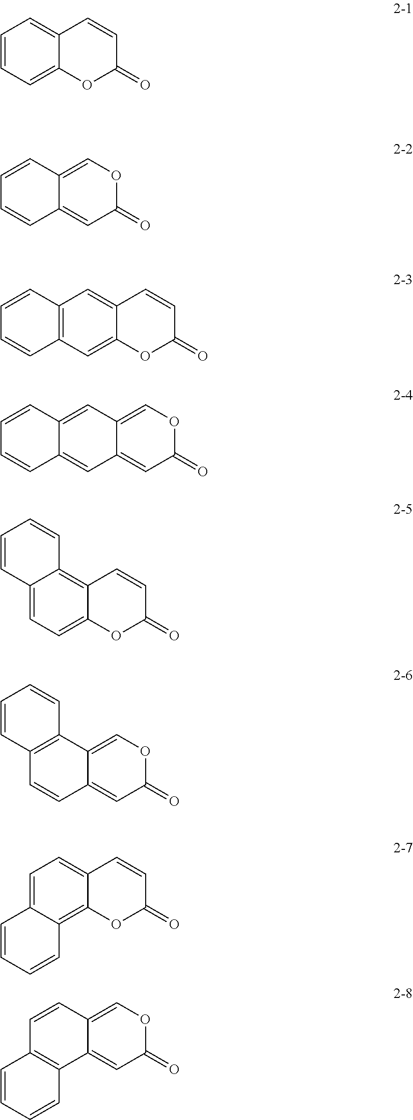

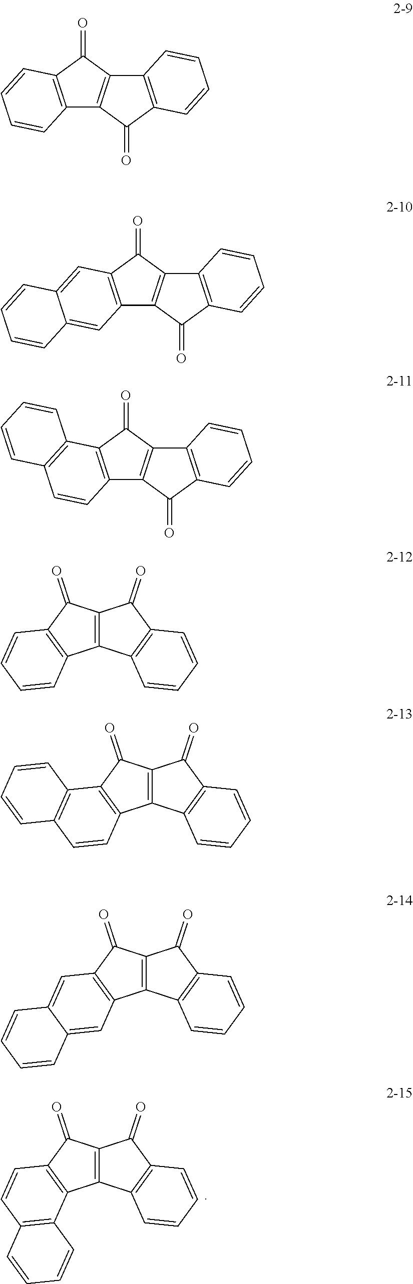

14. The organic light-emitting device of claim 1, wherein the fluorescent compound is one of Compounds 1 to 8:

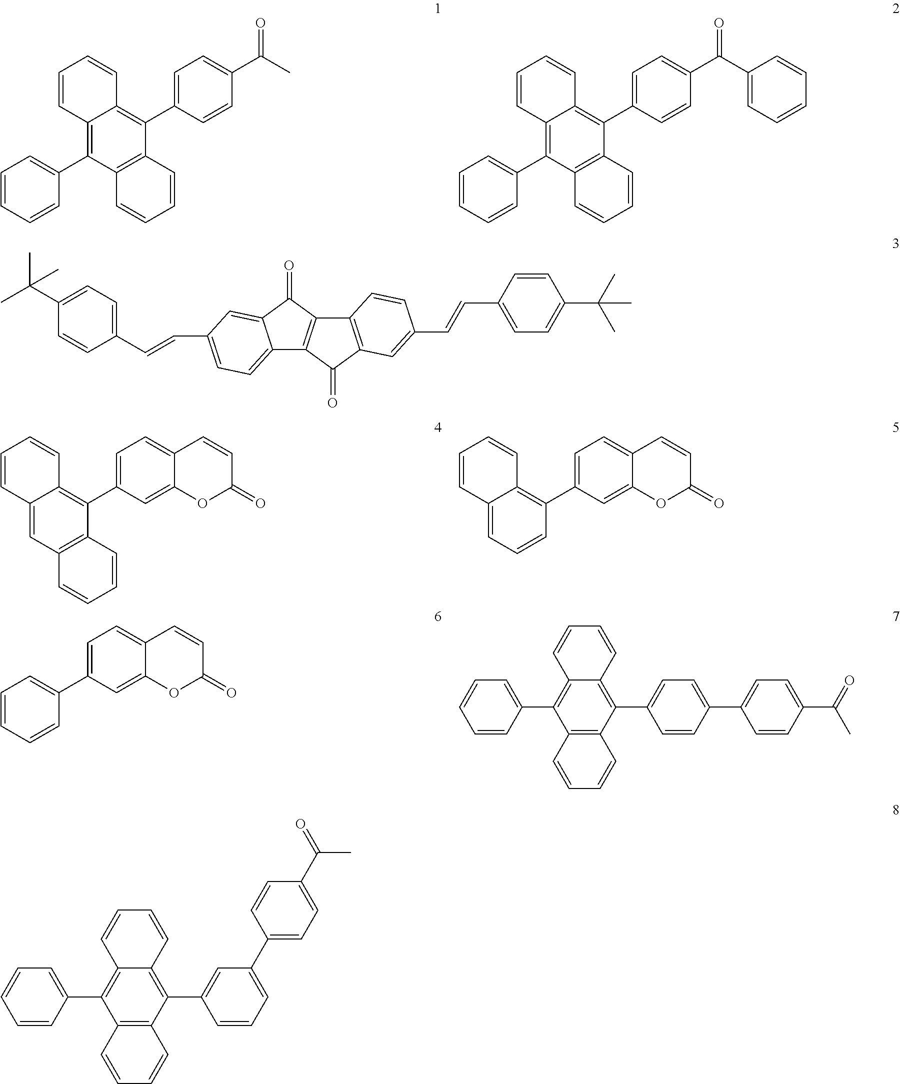

15. The organic light-emitting device of claim 1, wherein the emission layer comprises the fluorescent compound.

16. The organic light-emitting device of claim 15, wherein, a ratio of an emission portion of the fluorescent light emitted by radiative energy transition of the exciton in the ¹π-π* excited state to the ground state, which is transferred from the ³n-π* excited state of the fluorescent compound to the ¹π-π* excited state of the fluorescent compound via reverse intersystem crossing, to a total emission portion of light emitted from the emission layer is at least 90%.

17. The organic light-emitting device of claim 15, wherein the emission layer further comprises a host.

18. The organic light-emitting device of claim 1, wherein the organic layer comprises a hole transport region disposed between the first electrode and the emission layer, and an electron transport region disposed between the emission layer and the second electrode,
wherein the hole transport region comprises a hole injection layer, a hole transport layer, an electron blocking layer, a buffer layer or a combination thereof, and
the electron transport region comprises a hole blocking layer, an electron transport layer, an electron injection layer, or a combination thereof.

19. A fluorescent compound, wherein
the fluorescent compound comprises a ³n-π*-to-¹π-π* energy transition from a ³n-π* excited state to a ¹π-π* excited state,
an energy level in a ¹n-π* excited state of the fluorescent compound is greater than an energy level in the ¹π-π* excited state of the fluorescent compound,
the fluorescent compound emits a fluorescent light by radiative energy transition of an exciton in the ¹π-π* excited state to a ground state,
“³” in the expression “³n-π*” indicates a triplet state, and “¹” in the expressions “¹n-π*” and “¹π-π*” indicates a singlet state, and
the energy level in the ¹n-π* excited state, the energy level in the ¹π-π* excited state, and the energy level in the ³n-π* excited state are each independently calculated by using a time dependent-Density Functional Theory method that is structurally optimized at a level of CAM-B3LYP/6-311+G(d,p), and
the fluorescent compound is not a coumarin-based compound represented by Formula 1′: 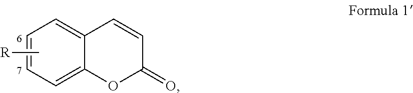
wherein, R in Formula 1′ is a C₆-C₅₀ aryl group positioned at the 6-position or 7-position of a coumarin ring in Formula 1′.

20. The fluorescent compound of claim 19, wherein the fluorescent compound comprises at least one carbonyl group.
